(19) United States
(12) Patent Application Publication
(10) Pub. No.: US 2026/0263456 A1
(43) Pub. Date: Sep. 10, 2026

(54) TADALAFIL GASTRIC-SOLUBLE RAPID-RELEASE SOLID DISPERSION, TABLET, AND USE THEREOF

(71) Applicant: Shenzhen Hanhui Pharmaceutical Technology Co., Ltd., Shenzhen (CN)

(72) Inventors: Allan Zijian Zhao, Shenzhen (CN); Yunping Mu, Shenzhen (CN); Fanghong Li, Shenzhen (CN); Zhuang Li, Shenzhen (CN); Haoheng Qiu, Shenzhen (CN); Jie Gao, Shenzhen (CN); Jianqin Yang, Shenzhen (CN); Aolu Liu, Shenzhen (CN)

(73) Assignee: Shenzhen Hanhui Pharmaceutical Technology Co., Ltd., Shenzen, GD (CN)

(21) Appl. No.: 19/166,260

(22) PCT Filed: Sep. 5, 2023
(86) PCT No.: PCT/CN2023/117058

§ 371 Date: Sep. 17, 2025

(30) Foreign Application Priority Data
Mar. 20, 2023 (CN) 202310292525.6

(51) Int. Cl. A61K 31/4985; A61K 9/20; A61P 9/12

(52) U.S. Cl. CPC A61K 31/4985; A61K 9/2013; A61K 9/2027; A61K 9/2031; A61K 9/2054; A61P 9/12

(57) ABSTRACT

A tadalafil gastric-soluble rapid-release solid dispersion, characterized by comprising tadalafil having a structure as shown in the following formula (I). An X-ray powder diffraction (XRPD) pattern of the solid dispersion is as shown in FIG. 5. There are no characteristic diffraction peaks at the following 20 angles (±0.2)°: 7.3°, 10.6°, 12.6°, 14.6°, 18.5°, 21.8°, and 24.3. Tadalafil is present in the solid dispersion in an amorphous form. The solid dispersion can be used to treat pulmonary hypertension, cardiac hypertrophy, erectile dysfunction, and for anti-fibrosis.

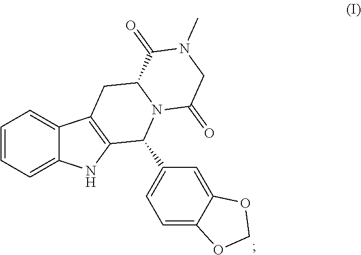 [SMILES: CN1CC(=O)N2[C@H](c3ccc4c(c3)OCO4)c3[nH]c4ccccc4c3C[C@@H]2C1=O]

### CROSS-REFERENCE TO RELATED APPLICATIONS

This application claims priority to a Chinese patent application filed with the China Patent Office on Mar. 20, 2023, under application No. 202310292525.6, titled “A Tadalafil Gastric Soluble Rapid Release Solid Dispersion, Tablet, and Its Application.” entire contents of this application are incorporated herein by reference.

### TECHNICAL FIELD

This application pertains to the field of pharmaceutical formulation technology, specifically focusing on a solid dispersion of Tadalafil designed for rapid gastric solubility and release. It also details the preparation method and applications of Tadalafil rapid-release tablets.

### BACKGROUND OF THE INVENTION

Signal transduction therapy is a significant area of focus in contemporary drug research. Cyclic guanosine monophosphate (cGMP) is a crucial hydrophilic second messenger that internalizes information conveyed by intercellular messengers and also functions as an autocrine signal. Currently, due to its ability to specifically hydrolyze cGMP and its role in regulating physiological processes such as smooth muscle contraction and relaxation, type 5 phosphodiesterase (PDE5) has emerged as a significant target for the treatment of erectile dysfunction and cardiovascular diseases. Common PDE5 inhibitors used in clinical practice include Tadalafil, Sildenafil, and Vardenafil. These medications are considered first-line treatments for erectile dysfunction and pulmonary arterial hypertension. Among them, Tadalafil is the only long-acting PDE5 inhibitor that exhibits a strong affinity for and selectively targets PDE5, and it has a wide range of applications. However, the issues of poor water solubility and low bioavailability of Tadalafil remain unresolved, which somewhat limits its clinical application and necessitates further in-depth research.

Fibrosis is a chronic, progressive disease characterized by the formation of scar tissue in various organs. It can affect multiple vital organs, including the lungs, liver, and kidneys. Due to the absence of effective treatment options, severe fibrous scars can compromise the structure and function of organ tissue, ultimately resulting in organ failure and posing a significant threat to human life and health. According to the latest real-time statistics from the World Health Organization (WHO), the number of confirmed COVID-19 cases worldwide has surpassed hundreds of millions. This situation has escalated into a global public health emergency, triggering a significant crisis affecting life, health, safety, and the economic and social order on a global scale. Research has found that patients with severe and critically ill COVID-19 infections experience significant inflammation and damage to their lungs, characterized by extensive infiltration of inflammatory cells and proliferation of fibrous tissue, ultimately leading to fibrosis. Consequently, the incidence of pulmonary fibrosis is expected to increase dramatically worldwide. However, the only clinically available treatment options at present are Nintedanib (BIBF 1120) and Pirfenidone, which can only alleviate certain symptoms. There is an urgent need to develop new antifibrotic medications.

It has been reported that liver fibrosis is a severe scar repair response that occurs after liver parenchymal cells are damaged due to viral hepatitis, fatty liver disease, autoimmune disorders, and other factors. This condition is caused by the excessive synthesis of extracellular matrix (ECM) in the interstitial spaces of liver tissue, or by a significant reduction in its degradation. This imbalance leads to the excessive deposition of ECM within liver cells, resulting in a high incidence rate and mortality associated with the condition. It has been reported that tissue and organ fibrosis is a significant contributor to the onset and progression of numerous diseases, potentially leading to disability and death, and affecting nearly a quarter of the global population. The total annual incidence rate of major fibrosis-related diseases is 4,968 per 100,000 people, including 3,400 per 100,000 people with liver fibrosis. It is important to note that, in comparison to the lethality of cirrhosis and liver cancer, multiple studies have confirmed that liver fibrosis is a reversible pathological process. If effective drug intervention is administered promptly during the early stages of the disease, fibrotic tissue can be reversed. However, if left untreated, liver fibrosis may progress to cirrhosis and significantly increase the risk of developing liver cancer. This progression not only poses a serious threat to human life and health but also imposes a substantial economic burden on society and families. At present, the development of drugs for treating liver fibrosis is progressing slowly. In clinical practice, chemical agents such as steroids and pyridone are primarily used for symptomatic treatment. While their efficacy is relatively well-established, they are associated with significant side effects and the potential for drug resistance, which limits their clinical use. Researchers and clinicians are dedicated to developing effective, less toxic, and less resistance-prone drugs for liver fibrosis; however, there are currently no new therapeutic options available on the market.

Chronic kidney disease (CKD) refers to structural abnormalities of the kidneys-such as atrophy, cysts, deformities, and tumors- or renal dysfunction that persists for three months or longer. Renal fibrosis is the pathological manifestation of the final common stage of CKD. Excessive deposition of ECM is a hallmark of renal fibrosis, which disrupts the physiological function of the kidneys and ultimately leads to their failure. About 13.4% of the global population is affected by CKD, with 79% of these individuals in the advanced stages of the condition, relying on dialysis or renal replacement therapy to sustain their lives. As the lifestyle and dietary habits of the Chinese population evolve, the number of CKD patients continues to rise. Furthermore, due to the limited understanding of CKD among patients, many cases progress to the later stages of the disease. The complex pathogenesis of renal fibrosis hinders the advancement of treatment options, resulting in a lack of direct therapeutic drugs specifically targeting this condition on the market. Consequently, there is an urgent need to develop new treatment strategies for renal fibrosis.

Research has demonstrated that PDE5 is extensively expressed in bronchial and pulmonary vascular smooth muscle cells, renal glomeruli, mesangial cells, intramedullary collecting ducts, cortical tubules, hepatic stellate cells, myocardial cells, and the corpus cavernosum of the penis. In recent years, numerous studies have investigated the antifibrotic effects of PDE5 inhibitors; however, the conclusions drawn from this research have been inconsistent, and the underlying mechanisms remain unclear. This uncertainty is particularly attributed to the low solubility of traditional PDE5 inhibitors. Therefore, it is essential to clarify the role and mechanism of PDE5 inhibitors in fibrosis, innovate the development of new formulations of these inhibitors, and enhance drug bioavailability.

### SUMMARY OF THE INVENTION

Therefore, the technical challenge addressed by the present application is to provide a solid dispersion of tadalafil that is soluble in gastric conditions and exhibits rapid release. This formulation significantly enhances both the dissolution rate and peak concentration of the tadalafil solid dispersion.

The second technical challenge addressed by this application is the development of a Tadalafil gastric-soluble rapid-release tablet that can dissolve quickly in the stomach.

The third technical challenge addressed by this application is to outline the preparation method and application of the previously mentioned Tadalafil gastric-soluble rapid-release solid dispersion and Tadalafil gastric-soluble rapid-release tablets.

In order to address the aforementioned technical issues, the present application discloses a Tadalafil gastric-soluble rapid-release solid dispersion, which comprises Tadalafil with the structure illustrated in the following formula (I):

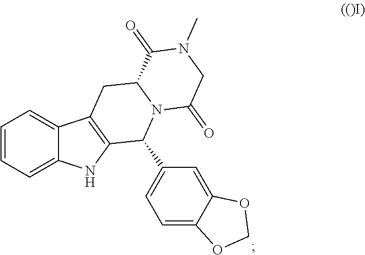 [SMILES: CN1CC(=O)N2[C@H](c3ccc4c(c3)OCO4)c3[nH]c4ccccc4c3C[C@@H]2C1=O]

The X-ray powder diffraction (XRPD) pattern of the solid dispersion is presented in FIG. 5. No characteristic diffraction peaks were observed at the following 2θ angles (±0.2°): 7.3°, 10.6°, 12.6°, 14.6°, 18.5°, 21.8°, and 24.3°. The absence of peaks corresponding to the characteristic diffraction data of Tadalafil (7.3°, 10.6°, 12.6°, 14.6°, 18.5°, 21.8°, and 24.3°) indicates that the active pharmaceutical ingredient is present in an amorphous form within the solid dispersion.

Specifically, the parameters for X-ray powder diffraction (XRPD) measurements are as follows: scanning angle: 3° to 70°; scanning speed: 8°/min; step size: 0.02°.

The TG-DSC spectrum of the Tadalafil gastric-soluble rapid-release solid dispersion is presented in FIG. 6. The differential scanning calorimetry (DSC) spectrum does not exhibit a characteristic melting absorption peak, nor is there an endothermic peak at 305.4° C. This indicates that Tadalafil exists in an amorphous form within the solid dispersion. The parameters for the DSC measurements are as follows: crucible material: aluminum; heating program: 25° C. to 350° C.; heating rate: 10° C./min; gas flow rate: 50 mL/min, N₂.

Specifically, the X-ray powder diffraction (XRPD) pattern of the Tadalafil gastric-soluble rapid-release solid dispersion is presented in FIG. 3. The pattern exhibits characteristic diffraction peaks at the following 2θ angles (±0.2°): 7.3°, 10.6°, 12.6°, 14.6°, 18.5°, 21.8°, and 24.3°.

Specifically, the parameters for the X-ray powder diffraction (XRPD) measurements are as follows: scanning angle: 3° to 70°; scanning speed: 8°/min; step size: 0.02°.

Specifically, the TG-DSC spectrum of the Tadalafil gastric-soluble rapid-release solid dispersion is presented in FIG. 4. Its differential scanning calorimetry (DSC) spectrum displays a characteristic endothermic peak at 305.4° C.

Specifically, the Tadalafil gastric-soluble rapid-release solid dispersion consists of Tadalafil, polymers, and surfactants.

The mass ratio of Tadalafil, polymer, and surfactant is 1:4-6:1:2.

Specifically, the Tadalafil gastric-soluble rapid-release solid dispersion consists of a mixture of one or more of the following: VA64, Soluplus, or HPMCP HP55.

Specifically, the Tadalafil gastric-soluble rapid-release solid dispersion, in which the surfactant consists of a mixture of one or more of sodium dodecyl sulfate (SDS) or tocopheryl polyethylene glycol succinate (TPGS).

Specifically, the Tadalafil gastric-soluble rapid-release solid dispersion consists of:
Tadalafil, VA64, and TPGS are combined in a mass ratio of 1:4:1. Alternatively,
Tadalafil, HPMC, VA64, and SDS were combined in a mass ratio of 1:4:2:1. Alternatively,
Tadalafil, HPMC, and SDS are combined in a mass ratio of 1:6:1. Alternatively,
Tadalafil, HPMC, SDS, and TPGS are combined in a mass ratio of 1:6:1:1.

This application also presents a method for preparing a gastric-soluble solid dispersion of tadalafil, which includes the following steps:

Mix the specified amount of Tadalafil, as indicated in Formula (I), with a solvent to create the initial suspension.

Add the specified amounts of the polymer and surfactant to the initial suspension to create a second suspension.

The second suspension is dried using a spray method.

The preparation method for Tadalafil gastric-soluble rapid-release solid dispersion specifically involves a solvent that consists of a mixture of one or more of the following: acetonitrile, acetone, tetrahydrofuran, dichloromethane, or ethanol.

Specifically, the preparation method for tadalafil stomach-soluble quick-release solid dispersion involves several parameters during the spray drying step: an inlet temperature of 80-100° C., an outlet temperature of 50-70° C., a peristaltic pump speed of 25-35%, a gas flow rate of 50-100 mm, N₂ as the gas source, a spray gun diameter of 0.6-0.8 mm, and a pumping speed of 100%.

Specifically, the preparation method for the gastric-soluble rapid-release solid dispersion of Tadalafil further includes the steps of ultrasound-assisted dissolution or heat-assisted dissolution of the second suspension.

This application also discloses a Tadalafil gastric-soluble rapid-release tablet, which comprises either the Tadalafil gastric-soluble rapid-release solid dispersion or the Tadalafil gastric-soluble rapid-release solid dispersion prepared by the specified method.

Specifically, the mass content of the solid dispersion in Tadalafil gastric-soluble rapid-release tablets ranges from 13.3% to 16.7% by weight.

This application also discloses the use of tadalafil gastro-soluble fast-release solid dispersions, or tadalafil gastro-soluble fast-release tablets, for the preparation of medications aimed at treating pulmonary arterial hypertension, myocardial hypertrophy, erectile dysfunction, or for antifibrotic purposes.

Specifically, the aforementioned anti-fibrosis treatments include at least one of the following: pulmonary fibrosis, liver fibrosis, or renal fibrosis.

The gastro-soluble, fast-release solid dispersion of tadalafil described in this application is achieved by selecting and optimizing the ratio of polymers and surfactants in the excipients. This formulation allows for the tadalafil to dissolve effectively in the stomach. Compared to the reference formulation of the original tadalafil drug, the solid dispersion demonstrates a significant increase in the dissolution rate in physiological saline and a higher peak blood concentration following the administration of the same dose. The gastro-soluble fast-release solid dispersion of tadalafil described in this application demonstrates superior efficacy at the same dosage compared to the original tadalafil formulation, providing the benefits of a lower dosage and enhanced effectiveness.

The gastro-resistant, fast-release solid dispersion of tadalafil described in this application exhibits low dosage and high efficacy in treating conditions such as pulmonary arterial hypertension and erectile dysfunction. It has also been found that this gastro-resistant, fast-release solid dispersion tablet is effective in treating pulmonary fibrosis, liver fibrosis, kidney fibrosis, and cardiac hypertrophy, while demonstrating low dosage and high efficacy. The preparation method is standardized and reliable, and the solid dispersion tablet exhibits good stability in its physicochemical properties, indicating its potential for development as a medication for the treatment of the aforementioned diseases.

### FIGURE DESCRIPTION

In order to enhance the clarity of this application, the following section provides a more detailed explanation based on specific implementation examples, accompanied by relevant figures.

FIG. 1 presents the full-wavelength UV scan of the tadalafil raw material drug in an acetonitrile medium.

FIG. 2 presents the linear establishment graph of the tadalafil raw material drug.

FIG. 3 presents the X-ray powder diffraction pattern of tadalafil.

FIG. 4 presents the TG-DSC spectrum of tadalafil.

FIG. 5 presents the X-ray powder diffraction graph of the novel solid dispersion of tadalafil, as prepared in Example 2.

FIG. 6 presents the Differential Scanning Calorimetry (DSC) curve of the novel solid dispersion of tadalafil, as prepared in Example 2.

FIG. 7 illustrates the dissolution curve of the reference formulation (Cialis) in pH 1.2 and water.

FIG. 8 illustrates the dissolution profile of the reference formulation (Cialis) in pH 1.2 with 0.5% Tween 80 and in water with 0.5% SLS.

FIG. 9 illustrates the dissolution profile of the tadalafil gastric-soluble controlled-release solid dispersion tablet (5 mg) prepared in Example 4, tested in pH 1.2 and water.

FIG. 10 illustrates the dissolution profile of the tadalafil gastric-soluble controlled-release solid dispersion tablet (2 mg) prepared in Example 5, tested in pH 1.2 and water.

FIG. 11 illustrates the average blood concentration-time curve for a 10 mg/kg reference formulation (Cialis) and the newly developed solid dispersion of tadalafil, which features gastric dissolution and controlled release.

FIG. 12 illustrates the effects of various doses of Cialis and a newly developed solid dispersion of tadalafil with controlled release on rat right ventricular systolic pressure (RVSP) after 14 days of treatment in pulmonary arterial hypertension (PAH) rats.

FIG. 13 illustrates the effects of various doses of Cialis and a newly developed solid dispersion of tadalafil with controlled release on rat right ventricular myocardial infarction (RVMI) after 14 days of treatment in pulmonary arterial hypertension (PAH) rats.

FIG. 14 displays standard images of H&E staining of pulmonary small arteries in various groups of PAH rats after 14 days of treatment with different doses of Cialis and a newly developed solid dispersion of tadalafil. The images are magnified at 100× (scale bar: 200 μm) and at 200× (scale bar: 100 μm).

FIG. 15 presents standard images of Masson's trichrome staining of lung tissue from various groups of PAH rats after 14 days of treatment with different doses of Cialis and a newly developed solid dispersion of tadalafil, magnified at 200× (scale bar: 100 μm).

FIG. 16 presents standard images of H&E staining of lung tissue from different groups of rats treated with various doses of Cialis and a new sustained-release formulation of tadalafil solid dispersions after 28 days. The images are magnified at 100× (scale bar: 200 μm) and at 200× (scale bar: 100 μm).

FIG. 17 displays standard images of Masson trichrome staining of lung tissue from different groups of rats treated with various doses of Cialis and a new sustained-release formulation of tadalafil solid dispersions after 28 days. The images are magnified at 100× (scale bar: 200 μm) and at 200× (scale bar: 100 μm).

FIG. 18 presents standard images of α-SMA immunohistochemistry in lung tissue from different groups of rats treated with various doses of Cialis and a new formulation of tadalafil enteric-coated immediate-release solid dispersions after 28 days. The images are magnified at 100× (scale bar: 200μ m).

FIG. 19 displays standard images of H&E staining of liver tissue from various groups of mice treated with Cialis and a new formulation of tadalafil enteric-coated immediate-release solid dispersions after 4 weeks, magnified at 100× (scale bar: 200 μm) and 200× (scale bar: 100 μm).

FIG. 20 displays standard images of Masson trichrome staining of liver tissue from various groups of mice treated with Cialis and a new formulation of tadalafil enteric-coated immediate-release solid dispersions after 4 weeks, magnified at 200× (scale bar: 100 μm).

FIG. 21 displays standard images of Sirius Red staining of liver tissues from various groups of mice treated with Cialis and tadalafil solid dispersion tablets for gastric release after 4 weeks, magnified at 100× (scale bar: 200 μm).

FIG. 22 displays standard images of α-SMA immunohistochemistry in liver tissues from various groups of mice treated with Cialis and tadalafil solid dispersion tablets for gastric release after 4 weeks, magnified at 100× (scale bar: 200 μm).

FIG. 23 presents standard images of H&E staining of kidney tissues from various groups of mice treated with Cialis and tadalafil solid dispersion tablets for gastric release after 7 days. The images are magnified at 100× (scale bar: 200 μm) and at 400× (scale bar: 50 μm).

FIG. 24 displays standard images of Masson's trichrome staining of kidney tissues from various groups of mice treated with Cialis and tadalafil solid dispersion tablets for gastric release after 7 days. The images are magnified at 100× (scale bar: 200 μm) and at 200× (scale bar: 100 μm).

FIG. 25 displays standard images of α-SMA immunohistochemistry in kidney tissues from various groups of mice treated with Cialis and tadalafil solid dispersion tablets for gastric release after 7 days, magnified at 100× (scale bar: 200 μm) and 200× (scale bar: 100 μm).

FIG. 26 illustrates the expression of fibrosis marker proteins α-SMA and FN in the renal tissue of mice with kidney fibrosis following 7 days of treatment with the new solid dispersion tablet formulation of Cialis and tadalafil.

### SPECIFIC IMPLEMENTATION METHODS

In the following examples of this application, for those that do not specify particular experimental steps or conditions, conventional experimental procedures or conditions, as described in the relevant literature, may be followed. Reagents or instruments not specified with a manufacturer are standard products that can be obtained through commercial sources.

In this application, the information regarding the tadalafil active pharmaceutical ingredient used in the following examples is presented in Table 1, while the details of the experimental reagents and equipment are provided in Tables 2 and 3, respectively.

TABLE 1
Information on the active pharmaceutical
ingredient compound Tadalafil

| Compound code | HZP |
|---|---|
| Batch | TDLF-2021011101 |
| Supplier | Ruyuan Dongyangguang |
| | Pharmaceutical Co., Ltd. |
| Other information | White powder |

TABLE 2
Experimental Reagents

| Reagent | Grade | Manufacturer | Batch Number |
|---|---|---|---|
| Water | Purified | Jing Tian (Shenzhen) | / |
| | Water | Food and Beverage | |
| | | Group Co., Ltd. | |
| Anhydrous | AR | China Petroleum & | 180161312B |
| Ethanol | | Chemical Corporation | |
| | | Nanjing Chemical | |
| | | Industry Co., Ltd. | |
| Acetone | AR | GENRAL | P1594060 |
| Tetrahydrofuran | AR | Beijing Dongfang Shibo | 20200116 |
| | | Fine Chemicals Co., Ltd. | |
| Acetonitrile | AR | SIGMA | WXBD1939V |
| Hydrochloric Acid | N/A | China National | 20200729 |
| | | Pharmaceutical Group | |
| | | Chemical Reagents Co., | |
| | | Ltd. | |
| Dihydrogen | N/A | Shanghai Shaoyuan | R1711304 |
| Potassium | | Reagent Co., Ltd. | |
| Phosphate | | | |
| Sodium | N/A | China Petroleum & | 180228711D |
| Hydroxide | | Chemical Corporation | |
| | | Nanjing Chemical | |
| | | Industry Co., Ltd. | |
| HPMC E5 | N/A | DOW | PD449197 |
| PVP K30 | N/A | BASF | G82196PTO |
| PEG4000 | N/A | JIANGXIN ALPHA | 20190402 |
| Soluplus | N/A | BASF | 20747936W0 |
| Kollidon VA64 | N/A | BASF | 89084288Q0 |
| HPMCAS LF | N/A | SHIN-ETSU | 8013007 |
| HPMCAS HF | N/A | ASHLAND | GSF-810003 |
| HPMCP HP55 | N/A | SHIN-ETSU | 8082127 |
| [symbol] | N/A | Aladdin | F1524011 |
| Kolliphor P188 | N/A | BASF | GND17221B |
| VE-TPGS | N/A | PMC | 1901010012 |
| HPC | N/A | SHIN-ETSU | 19476156P0 |

TABLE 3
Experimental Equipment

| Equipment Name | Manufacturer | Model |
|---|---|---|
| Analytical Balance | Sadolis, Germany | BT-125D |
| Differential | METTLER TOLEDO | DSC 3 |
| Scanning | | |
| Calorimeter (DSC) | | |
| Ultraviolet-Visible | Shimadzu Instrument Co., | UV2600 |
| Spectrophotometer | Ltd., Japan | |
| (UV-Vis) | | |
| Mini spay drier | Buchi | B-290 |
| Centrifugal Dryer | Genevac Inc SP Industry | Gene Vac HT-12 |
| Rotary Tablet Press | New Longli Machinery | ZP-10A |
| | Manufacturing Co., Ltd | |
| Dissolution Tester | Tianjin Tianda Tianfa | RC12AD |
| | Technology Co., Ltd | |

### Implementation Example 1: Tadalafil-Related Performance

### 1. Selection of Organic Solvents

A suitable amount of tadalafil was weighed and dissolved in various commonly used organic solvents to identify the most appropriate solvents for establishing UV methodology. The solvents utilized for preparing solid dispersions, along with the solubility of tadalafil, the active pharmaceutical ingredient, in different solvents, are presented in Table 4 below.

TABLE 4
Solubility of Tadalafil Active Pharmaceutical
Ingredient (API) in Various Solvents

| | | | | Is it | |
|---|---|---|---|---|---|
| | | | Solvent | completely | Solubility |
| Number | API/mg | Solvent | volume | dissolved(Y/N) | (mg/mL) |
| 1 | 9.57 | Acetonitrile | 0.2 mL | 25° C.(N) | 15.95 < S < 23.93 |
| | | | | 60° C.(N) | |
| | | | 0.4 mL | 25° C.(N) | |
| | | | | 60° C.(N) | |
| | | | 0.6 mL | 25° C.(Y) | |
| | | | | 60° C.(Y) | |
| 2 | 10.82 | Anhydrous | 0.2 mL | 25° C.(N) | S < 10.82 |
| | | ethanol | | 60° C.(N) | |
| | | | 0.4 mL | 25° C.(N) | |
| | | | | 60° C.(N) | |
| | | | 0.6 mL | 25° C.(N) | |
| | | | | 60° C.(N) | |
| | | | 0.8 mL | 25° C.(N) | |
| | | | | 60° C.(N) | |
| | | | 1.0 mL | 25° C.(N) | |
| | | | | 60° C.(N) | |
| 3 | 10.17 | Acetone | 0.2 mL | 25° C.(N) | 10.17 < S < 16.95 |
| | | | | 60° C.(N) | |
| | | | 0.4 mL | 25° C.(N) | |
| | | | | 60° C.(N) | |
| | | | 0.6 mL | 25° C.(N) | |
| | | | | 60° C.(N) | |
| | | | 1.0 mL | 25° C.(Y) | |
| | | | | 60° C.(Y) | |
| 4 | 9.53 | Tetrahydrofuran | 0.2 mL | 25° C.(N) | 23.83 < S < 47.65 |
| | | | | 60° C.(N) | |
| | | | 0.4 mL | 25° C.(Y) | |
| | | | | 60° C.(Y) | |
| 5 | 4.69 | Dichloromethane | 0.2 mL | 25° C.(N) | S < 10.81 |
| | | | | 60° C.(N) | |
| | | | 0.4 mL | 25° C.(N) | |
| | | | | 60° C.(N) | |
| | | | 0.6 mL | 25° C.(N) | |
| | | | | 60° C.(N) | |
| | | | 0.8 mL | 25° C.(N) | |
| | | | | 60° C.(N) | |
| | | | 1.0 mL | 25° C.(N) | |
| | | | | 60° C.(N) | |
| 6 | 11.25 | pH 6.8 | 1.0 mL | 25° C.(N) | S < 8.04 |
| | | | | 60° C.(N) | |
| | | | 1.4 mL | 25° C.(N) | |
| | | | | 60° C.(N) | |

### 2. Establishment of UV Methodology

### UV Detection Wavelength

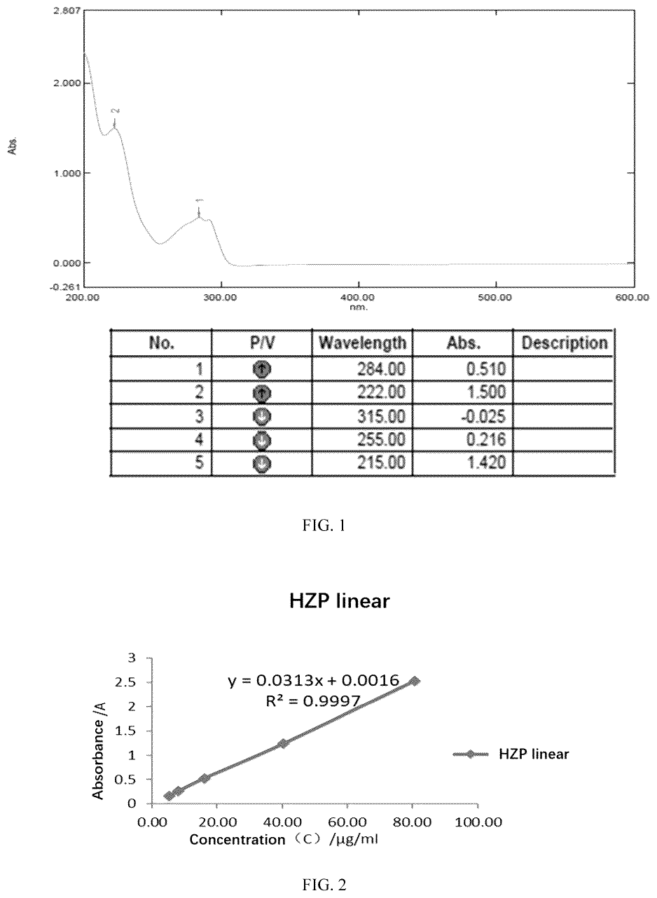

A stock solution with a concentration of 0.5 mg/mL was prepared by diluting it with acetonitrile and then further diluted to a concentration of 0.015 mg/mL. A full wavelength scan was conducted in the range of 200 to 600 nm, with the results presented in Appendix FIG. 1. From FIG. 1, it is evident that a wavelength of 284 nm was selected for absorbance measurement, and the solid dispersion was developed using the UV method.

### Linearity and Range

Using the stock solution diluted with acetonitrile, test solutions were prepared at concentrations of 5, 8, 16, 40, and 80 μg/mL. The absorbance was measured at a wavelength of 284 nm, resulting in the following concentration-absorbance linear regression equation (see FIG. 2): y=0.0313x+0.0016, R²=0.9997. The results presented in FIG. 2 indicate that the solution concentrations range from 5.38 to 80.72 μg/mL, with R²>0.999, demonstrating excellent linearity.

### 3. Determination of Differential Scanning Calorimetry (DSC) and X-ray Powder Diffraction (XRPD) for Tadalafil Raw Material

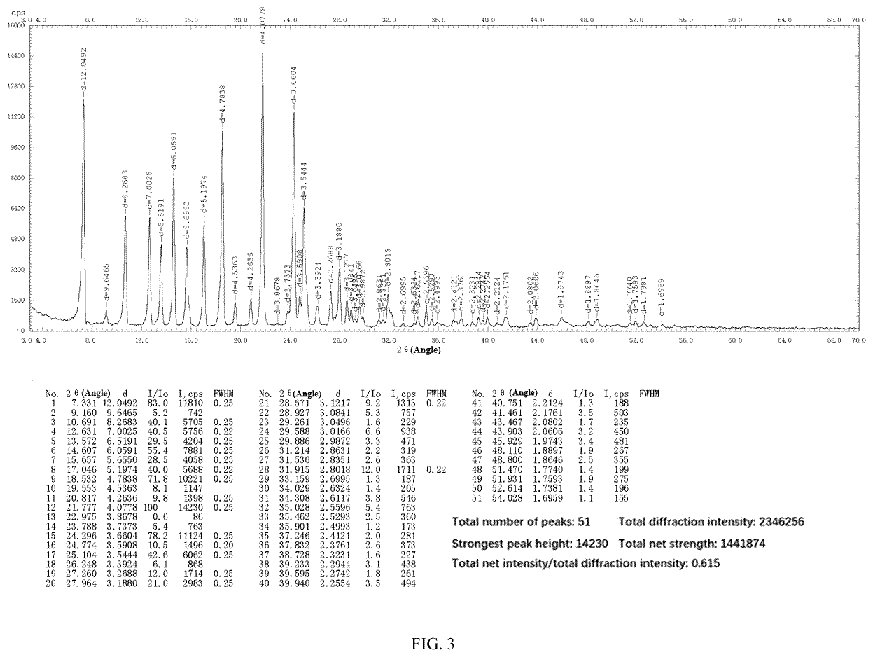

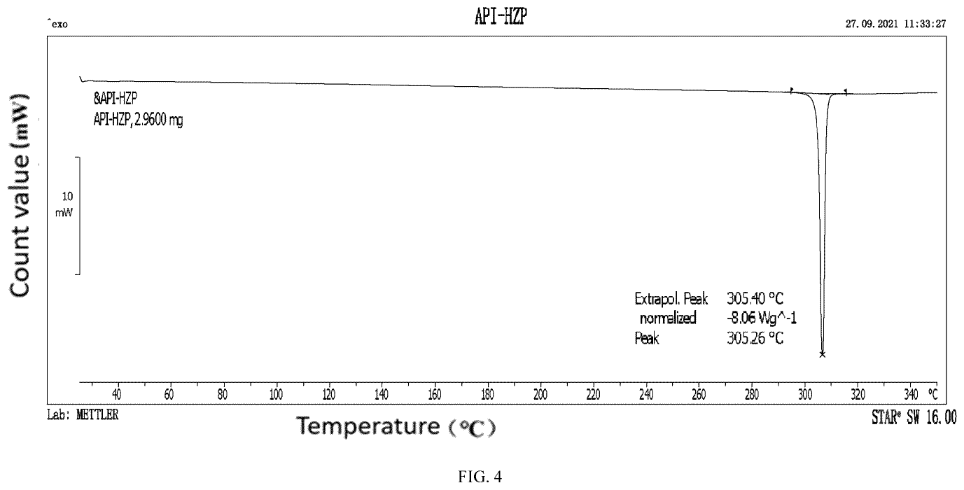

Differential Scanning Calorimetry (DSC) and X-ray Powder Diffraction (XRPD) tests for tadalafil raw material were conducted over a temperature range of 25 to 350° C., with the results presented in FIGS. 3 and 4. The data indicate that tadalafil exhibits a characteristic endothermic peak at 305.4° C., suggesting that the melting point of the raw material is approximately 305.4° C. Given the high melting point of tadalafil, the feasibility of preparing solid dispersions using the hot melt extrusion method is limited; therefore, the solvent method, specifically the spray drying technique, is preferred for the preparation of solid dispersions.

### Implementation Example 2: Gastro-Resistant Fast-Release Solid Dispersion of Tadalafil

### 1. Selection of Polymers and Surfactants

In this implementation example, a micro-scale rotary evaporation method was employed to screen various types of polymers and surfactants, specifically in the context of the active pharmaceutical ingredient (API) ratio of polymer to surfactant.

Acetone was utilized to prepare a stock solution of the active pharmaceutical ingredient at a concentration of 5 mg/mL, a polymer stock solution at 10 mg/mL, and a surfactant stock solution at 10 mg/mL. These solutions were evenly mixed in two identical portions, then centrifuged and dried to create the solid dispersion.

To each sample tube, 1 mL of pH 1.0 and pH 6.8 media was added, and the samples were vortexed at room temperature to ensure complete dispersion. Samples were collected at 5 minutes and 30 minutes, then centrifuged, diluted, and the absorbance was measured to calculate solubility. The results are presented in Table 5.

TABLE 5
Solubility of Polymer and Surfactant: Screening Preparation
of Solid Dispersion in pH 1.0 and pH 6.8 Media

| | | | | | pH 1.0(Solubility | | pH 6.8(Solubility | |
|---|---|---|---|---|---|---|---|---|
| | Raw | | | | mg/mL) | | mg/mL) | |
| Number | material | Polymer | Surfactant | Proportion | 5 min | 30 min | 5 min | 30 min |
| API | | | | 2 mg/mL | 0.002 | | 0.027 | |
| SD-1 | API | Soluplus | SDS | 1:3:0.2 | 0.162 | 0.502 | 0.298 | 0.398 |
| SD-2 | | | P188 | 1:3:1 | 0.385 | 0.390 | 0.296 | 0.547 |
| SD-3 | | | TPGS | 1:3:1 | 0.369 | 0.569 | 0.173 | 0.613 |
| SD-4 | | VA 64 | SDS | 1:3:0.2 | 0.097 | 0.044 | 0.077 | 0.060 |
| SD-5 | | | P188 | 1:3:1 | 0.170 | 0.094 | 0.212 | 0.173 |
| SD-6 | | | TPGS | 1:3:1 | 0.251 | 0.134 | 0.272 | 0.152 |
| SD-7 | | | | 1:4:1 | 0.676 | 0.569 | 0.518 | 0.510 |
| SD-8 | | | | 1:5:1 | 1.026 | 0.627 | 0.811 | 0.569 |
| SD-9 | | | | 1:5:1.5 | 1.044 | 0.838 | 0.904 | 0.779 |
| SD-10 | | HPMCAS | SDS | 1:3:0.2 | 0.025 | 0.021 | 0.060 | 0.163 |
| SD-11 | | LF | P188 | 1:3:1 | 0.024 | 0.019 | 0.063 | 0.069 |
| SD-12 | | | TPGS | 1:3:1 | 0.175 | 0.186 | 0.195 | 0.263 |
| SD-13 | | HPMCP | TPGS | 1:3:1 | 0.149 | 0.153 | 0.214 | 0.645 |
| SD-14 | | HP55 | SDS | 1:3:0.2 | 0.047 | 0.045 | 0.415 | 0.512 |
| SD-15 | | | P188 | 1:3:1 | 0.027 | 0.025 | 0.304 | 0.484 |

In summary, the analysis of solid dispersion results indicates that the polymers VA64, Soluplus, and HPMCP HP55, along with the surfactants SDS and TPGS, all enhance the solubility of the compound. Based on a comprehensive evaluation, the formulations for the fast-release tablets can be selected as follows:—Active Pharmaceutical Ingredient (API):VA64:TPGS=1:4:1—API:HPMC:VA64:SDS=1:4:2:1—API:HPMC:SDS=1:6:1-API:HPMC:SDS:TPGS=1:6:1:1.

### 2. Preparation of Solid Dispersions

### Spray Drying Sample Preparation

Taking the preparation method of batch HZP-21101101 as an example, weigh the specified amount of active pharmaceutical ingredient (API) and dissolve it in acetone. Next, dissolve the designated amounts of Kollidon VA64 and TPGS in acetone. Mix the two solutions thoroughly and dissolve them using ultrasonic treatment or by heating to 60° C. Proceed with spray drying (the spray drying process is detailed in Table 6 below) to prepare the solid dispersion with a ratio of API:VA64:TPGS=1:4:1. The formulation and dissolution performance results of the solid dispersion are presented in Tables 7 and 8 below.

TABLE 6
Spray Drying Process (Equipment Model: BUCHI B-290)

| | | | |
|---|---|---|---|
| Inlet | 90 | Outlet temperature/ | 60 |
| temperature/° C. | | ° C. | |
| Pump speed/% | 30 | Airflow Rate/mm | 100 |
| Spray | 0.7 | Pumping speed/% | 100 |
| nozzle/mm | | | |

| | |
|---|---|
| Phenomenon | The separator is in good condition |
| | and does not haveany adhesive. |

TABLE 7
Formulation and Preparation of Solid Dispersions

| | |
|---|---|
| Compound name | HZP |
| Batch Number of | HZP-21101101 |
| Solid Dispersion | |
| ASD prescription | API:VA64:TPGS |
| Proportion (w/w) | 1:4:1 |
| API Drug Loading | 16.67% |
| Capacity | |
| Preparation method | Spray drying |
| Best solubility | 0.712 mg/mL(Acid to Alkali |
| | Conversion, pH 6.8) |

TABLE 8
Solubility Data of Solid Dispersions in pH 1.0 and pH 6.8 Media

| | | Solid dispersion(API:VA64:TPGS = 1:4:1) |
|---|---|---|
| Medium | Time/min | Con(mg/mL) |
| pH 1.0 | 30 | 1.398 |
| | 60 | 1.102 |
| pH 6.8 | 120 | 0.712 |
| | 240 | 0.627 |
| | 360 | 0.378 |

The solubility data for the solid dispersion formulation meets the specifications for dissolution conditions of 5 mg and 20 mg.

### 3. Determination of DSC and XRPD for Solid Dispersions

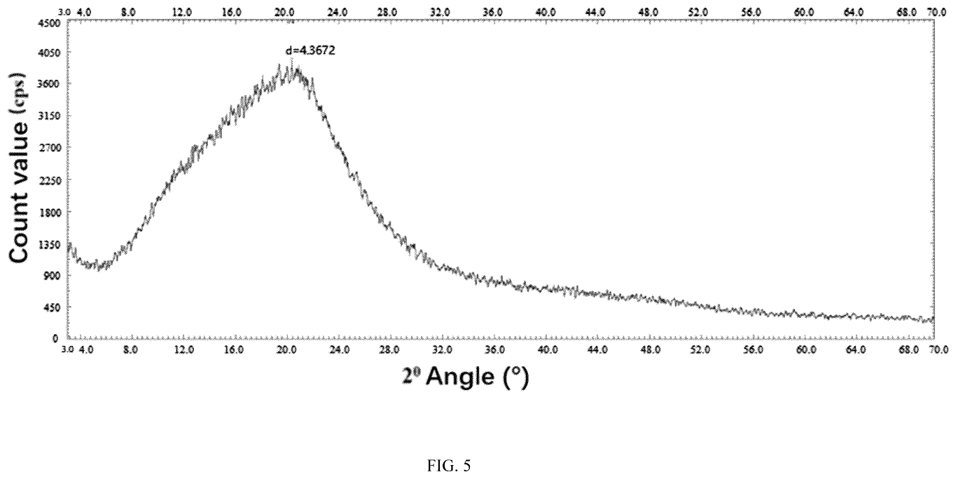

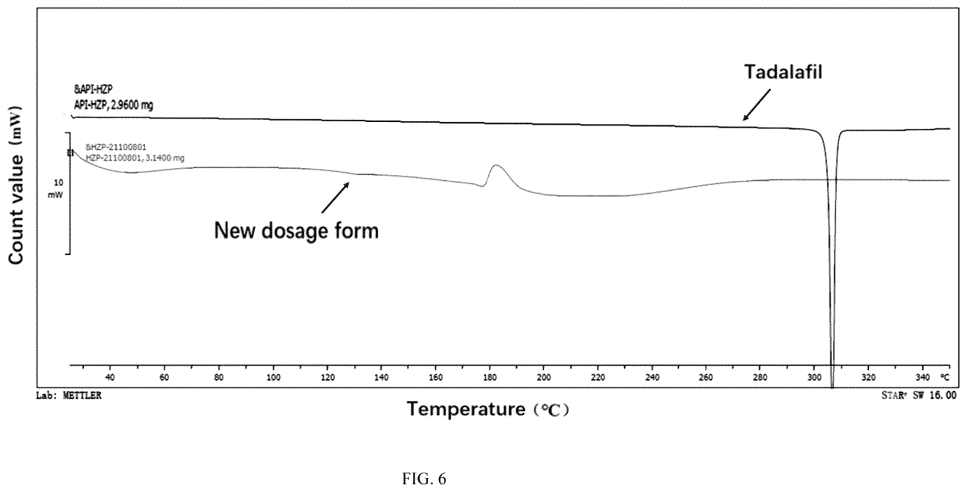

DSC curves and XRPD tests were performed on batch sample HZP-21101101 over a temperature range of 25 to 350° C., with the results presented in FIGS. 5 and 6. The DSC results for the solid dispersion indicate the absence of a characteristic endothermic peak at the melting point of tadalafil (305.4° C.). Additionally, the XRPD results reveal no distinct diffraction peaks for tadalafil, suggesting that the active pharmaceutical ingredient is not present in its crystalline form within the solid dispersion formulation. This implies that the active pharmaceutical ingredient exists in an amorphous state within the solid dispersion.

### Implementation Example 3: Dissolution Curve Measurement of the Reference Formulation (Cialis)

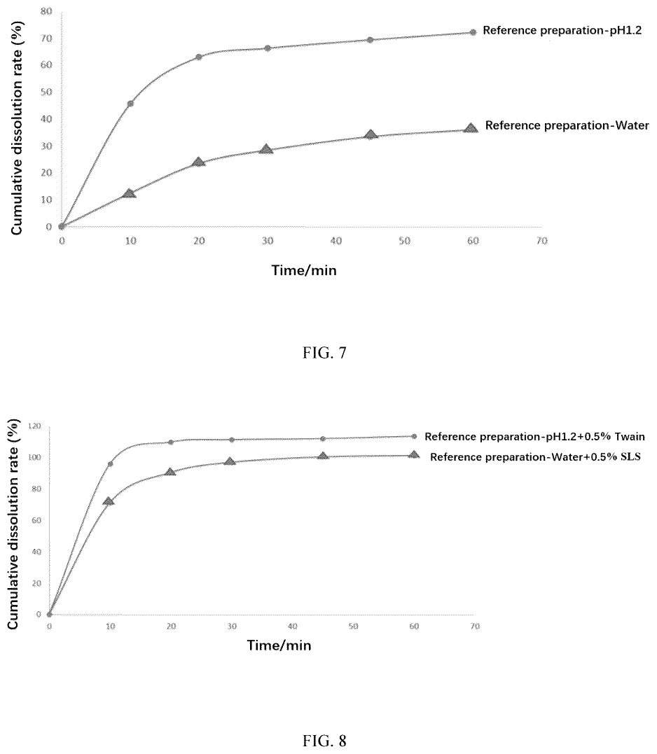

In this example, the dissolution curve of the reference formulation (Cialis) is analyzed. The dissolution methods and the dissolution degrees of the reference formulation (Cialis) across different systems are presented in Tables 9-11. The dissolution curve of the reference formulation in pH 1.2 and water is depicted in FIG. 7, while the dissolution degree of the reference formulation in pH 1.2 with 0.5% Tween 80 and water with 0.5% SLS is illustrated in Appendix FIG. 8.

TABLE 9
Dissolution methods

| | | | |
|---|---|---|---|
| Method | Slurry method | Temperature | 37° C. ± 0.5° C. |
| Rotational | 50 rpm | Sample volume | 5 mL (Fluid |
| speed | | | replacement 5 mL) |

| | |
|---|---|
| Sampling time | 5 min, 15 min, 30 min, 45 min, 60 min, 90 min, 120 min |
| Dissolution | pH 1.2, Water, pH 1.2 + 0.5% Tween80, |
| medium | Water + 0.5% SLS |
| Sample | Reference formulation specification: 5 mg |
| information | |

TABLE 10
Dissolution of Reference Formulations in pH 1.2 and Water

Cumulative dissolution rate %

| Time/min | Medium-pH 1.2 | Medium-aqueous solution |
|---|---|---|
| 10 | 12.52 | 45.76 |
| 20 | 23.52 | 63.04 |
| 30 | 28.47 | 66.33 |
| 45 | 33.47 | 69.45 |
| 60 | 36.06 | 72.18 |

TABLE 11
Dissolution of Reference Formulations in pH 1.2
with 0.5% Tween 80 and Water with 0.5% SLS

Cumulative dissolution rate %

| Time/min | pH 1.2 + 0.5% Tween 80 | Water + 0.5% SLS |
|---|---|---|
| 10 | 71.20 | 95.89 |
| 20 | 90.28 | 109.73 |
| 30 | 96.82 | 111.23 |
| 45 | 100.23 | 111.91 |
| 60 | 101.11 | 113.41 |

Note:
A dissolution rate exceeding 100% may result from the absence of a reference standard.

The raw material was used for comparison without proper calibration, which could lead to inflated data; however, this does not impact the trend assessment.

The results indicate that the reference formulation exhibits a lower dissolution rate in a medium without surfactants and does not fully dissolve. However, when surfactants are introduced into the medium, the formulation dissolves rapidly.

### Implementation Example 4: Development of a Solid Dispersion Formulation

This implementation example focuses on the development of a gastro-soluble, rapid-release tablet based on the solid dispersion prepared in Implementation Example 2. The tablet specification is 5 mg, and the formulation composition of the tablet is presented in Table 12 below.

TABLE 12
Prescription Composition

Tadalafil Rapid Release Tablets

| | Prescription | | Unit | |
|---|---|---|---|---|
| Component | composition | Proportion | dose | |
| Solid | API | 16.7% | 5 | mg |
| component | VA64 | | 20 | mg |
| | TPGS | | 5 | mg |
| Filler | Lactose | 67.8% | 122.0 | mg |
| | Microcrystalline | 5.5% | 9.9 | mg |
| | cellulose | | | |
| Disintegrants | Croscarmellose | 5.5% | 9.9 | mg |
| | Sodium | | | |
| Glidants | Colloidal silica | 3% | 5.4 | mg |
| Lubricant | Sodium stearyl | 0.5% | 0.9 | mg |
| | fumarate | | | |
| | Magnesium | 1% | 1.8 | mg |
| | stearate | | | |
| / | Total weight | 100% | 180 | mg |

The preparation process for the enteric-coated fast-release tablet described in this embodiment includes:
1) Weigh the prescribed amount of solid dispersion and silica. Mix them evenly using a 40-mesh sieve. Then, weigh the remaining excipients and mix them uniformly using an incremental method with equal amounts, also through a 40-mesh sieve.
Compress the mixture into tablets, adhering to the specified theoretical tablet weight.
Conduct dissolution curve testing on the compressed tablets.

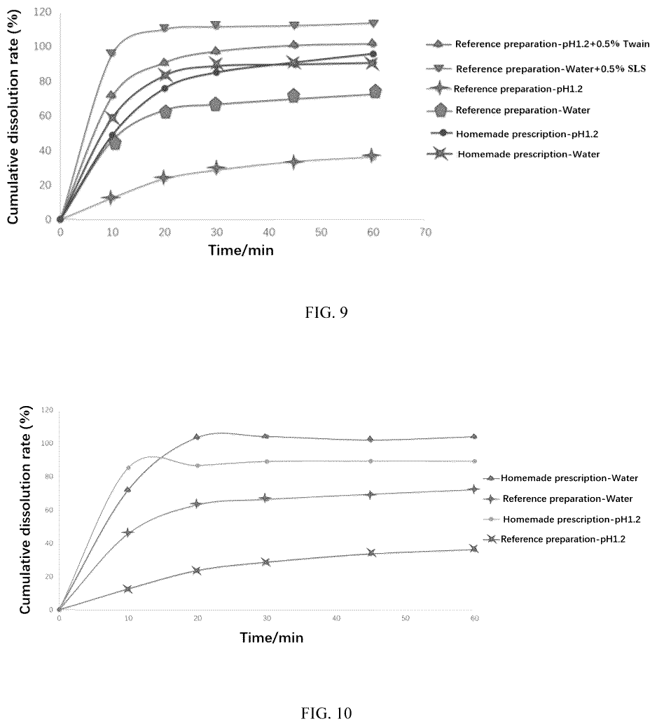

In this embodiment, the method for determining the dissolution curve of the solid dispersion tablet and the corresponding dissolution results is presented in Tables 13 and 14. The dissolution curves of the tablets in both water and pH 1.2 media are illustrated in FIG. 9.

TABLE 13
Conditions for Determining the Dissolution Curve

| | | | |
|---|---|---|---|
| Method | Slurry method | Temperature | 37° C. ± 0.5° C. |
| Rotational | 50 rpm | Sample volume | 5 mL (Fluid |
| speed | | | replacement 5 mL) |

| | |
|---|---|
| Sampling time | 5 min, 15 min, 30 min, 45 min, 60 min |
| Dissolution | pH 1.2, Water |
| medium | |
| Sample | Homemade Product Specifications: 5 mg, 6 Tablets |
| information | |

TABLE 14
Dissolution of Solid Dispersion Prescription
Tablets in pH 1.2 and Water

Cumulative dissolution rate %

| Time/min | Medium-pH 1.2 | Medium-aqueous solution |
|---|---|---|
| 10 | 48.84 | 58.89 |
| 20 | 75.59 | 83.08 |
| 30 | 84.75 | 88.62 |
| 45 | 90.51 | 89.49 |
| 60 | 95.50 | 90.36 |

Result Analysis: As illustrated in FIG. 9, the dissolution curves of the solid dispersion tablet formulation in both water and pH 1.2 media closely align with the dissolution curve data of the reference formulation containing surfactant in these same media.

### Implementation Example 5: Development of Solid Dispersion Formulations

This implementation example focuses on the development of a gastro-soluble, rapid-release tablet based on the solid dispersion prepared in Implementation Example 2. The tablet specification is 2 mg, and the formulation composition of the tablet is presented in Table 15 below.

TABLE 15
Prescription Composition

Tadalafil Rapid Release Tablets

| | Prescription | | Unit | |
|---|---|---|---|---|
| Component | composition | Proportion | dose | |
| Solid | API | 13.3% | 2 | mg |
| component | VA64 | | 8 | mg |
| | TPGS | | 2 | mg |
| Filler | Lactose | 66.7% | 60.03 | mg |
| | Microcrystalline | 10% | 9 | mg |
| | cellulose | | | |
| Disintegrants | Croscarmellose | 5% | 4.5 | mg |
| | Sodium | | | |
| Glidants | Colloidal silica | 3% | 2.7 | mg |
| Lubricant | Sodium stearyl | 1% | 0.9 | mg |
| | fumarate | | | |
| | Magnesium | 1% | 0.9 | mg |
| | stearate | | | |
| / | Total weight | 100% | 90 | mg |

The preparation process for the enteric-coated fast-release tablet in this embodiment includes:
1) Weigh the prescribed amount of solid dispersion and silica. Mix them evenly using a 40-mesh sieve. Then, weigh the remaining excipients and mix them uniformly using an equal amount incremental method, also through a 40-mesh sieve.
Compress the mixture into tablets according to the specified theoretical tablet weight.
Conduct dissolution curve testing on the compressed tablets.

In this embodiment, the method for determining the dissolution curve of the solid dispersion tablet and the corresponding dissolution results is presented in Tables 16 and 17. The dissolution curves of the tablets in both water and pH 1.2 media are illustrated in FIG. 10.

TABLE 16
Conditions for Determining the Dissolution Curve

| | | | |
|---|---|---|---|
| Method | Slurry method | Temperature | 37° C. ± 0.5° C. |
| Rotational | 50 rpm | Sample volume | 5 mL (Fluid |
| speed | | | replacement 5 mL) |

| | |
|---|---|
| Sampling | 5 min, 15 min, 30 min, 45 min, 60 min |
| time | |
| Dissolution | pH 1.2, Water |
| medium | |
| Sample | Homemade Product Specifications: 2 mg |
| information | |

TABLE 17
Dissolution of 2 mg Solid Dispersion Prescription
Tablets in pH 1.2 and Water

| | |
|---|---|
| Reference preparation | Rapid-release Tablets |

| Time/min | pH 1.2 | Water | pH 1.2 | Water |
|---|---|---|---|---|
| 10 | 12.5 | 45.8 | 85.2 | 71.8 |
| 20 | 23.5 | 63.0 | 86.5 | 103.3 |
| 30 | 28.5 | 66.3 | 88.9 | 103.8 |
| 45 min | 33.5 | 69.5 | 89.3 | 101.8 |
| 60 min | 36.1 | 72.2 | 89.1 | 103.8 |

FIG. 10 illustrates that the dissolution curve of the self-manufactured product with a specification of 2 mg outperforms the reference formulation in both pH 1.2 and water. In summary, the tadalafil gastric-soluble fast-release solid dispersion tablet developed in Example 2 of this application exhibits a superior dissolution rate compared to Cialis. The following section will provide a detailed explanation of the pharmaceutical applications of the tadalafil gastric-soluble fast-release solid dispersion tablet prepared in this example, along with specific experimental data.

### Experimental Example: Testing the Drug Activity of a New Gastro-Resistant, Fast-Release Solid Dispersion Tablet Formulation of Tadalafil

### 1. Pharmacokinetic Study of a Gastro-Resistant, Fast-Release New Formulation of Solid Dispersion Tablets Containing Tadalafil

### Experimental Materials

Reagents: Tadalafil gastro-resistant fast-release solid dispersion tablets (2 mg) were prepared as described in Example 5. Physiological saline and pentobarbital sodium were obtained from China National Pharmaceutical Group Co., Ltd., while acetonitrile, formic acid, and methanol were sourced from China Baolingwei Chemical Reagent Co., Ltd.
Instruments: Ultra-Fast Liquid Chromatography-Tandem Mass Spectrometry (4000 QTRAP LC-MS/MS); Mass Spectrometer: AB Sciex API 4000 QTRAP; Liquid Chromatograph: Shimadzu LC-20ADXR.
Experimental Animals: Male SPF-grade Sprague-Dawley (SD) rats, weighing between 180 and 200 grams, were obtained from Beijing Vital River Laboratory Animal Technology Co., Ltd. The experimental animal license number is SCXK (Beijing)-2002-0011. The rats were housed at the Experimental Animal Center of South China University of Technology, where they were maintained at a temperature of (22±2° C.), a humidity level of (60±5) %, and a 12-hour light-dark cycle.

### Experimental Method

Six healthy male SD rats were fasted for 12 hours with free access to water. Following the fasting period, the rats were administered a gastric gavage of Cialis (10 mg/kg) and a new formulation of tadalafil in solid dispersion tablets. Blood samples were collected from the orbital venous plexus at various time points: 0 minutes, 5 minutes, 15 minutes, 30 minutes, 60 minutes, 90 minutes, 120 minutes, 180 minutes, 240 minutes, 360 minutes, 8 hours, 10 hours, 12 hours, 24 hours, 36 hours, and 48 hours post-administration. Acetonitrile was added to the samples, which were then centrifuged to obtain the supernatant for liquid chromatography-mass spectrometry (LC-MS) analysis.

Chromatographic Conditions: The chromatography was conducted using a Thermo Hypersil-Keystone C18 column (particle size: 5 μm; dimensions: 50 mm×2.1 mm). The injection volume was 1 μL, with a flow rate of 0.3 mL/min and a column temperature set at 40° C. The mobile phase comprised water (0.1% formic acid) and acetonitrile, utilizing a gradient elution as follows: 10% acetonitrile at 0 minutes; 10% at 1 minute; 90% at 4 minutes; 90% at 6 minutes; and returning to 10% at 7 minutes.

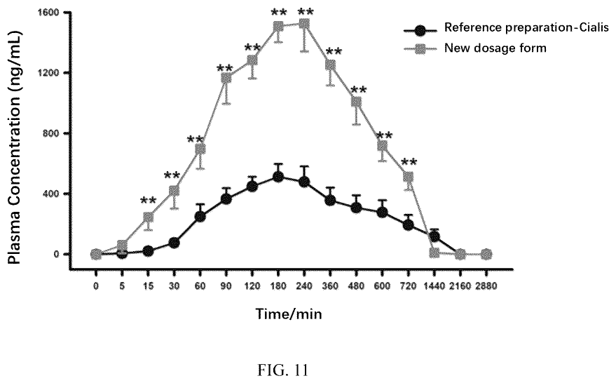

Mass spectrometry conditions: Multiple reaction monitoring (MRM) was employed with electrospray ionization (ESI). The dwell time was set to 200 ms, and the detected ions ranged from m/z 390.2 to 337.2. Each sample underwent a stabilization period of 2 minutes. The blood concentration data were analyzed using WinNonlin 6.3 software to obtain pharmacokinetic parameters, resulting in an average blood concentration-time curve, as illustrated in FIG. 11.

### Experimental Results

After the oral administration of Cialis in male Sprague-Dawley (SD) rats, the drug concentration peaked at 3 hours, with a maximum concentration (Cₘₐₓ) of 512.2±85.1 ng/ml. In contrast, the new formulation of tadalafil in a solid dispersion tablet reached its peak concentration at 4 hours post-administration in male SD rats, with a maximum concentration (Cₘₐₓ) of 1527.3±185.3 ng/ml, which is nearly three times that of the original formulation.

### 2. Experiment on Pulmonary Arterial Hypertension (PAH) in Rats

### Experimental Materials

Reagents: The tadalafil gastro-resistant fast-release solid dispersion tablets (2 mg) were prepared as described in Example 5. Cialis was purchased from JD Pharmacy (Batch No.: D448743). Monocrotaline (MCT) was obtained from Sigma Chemical Company, USA. Heparin, uridine solution, xylene, and paraffin were purchased from China National Pharmaceutical Group Corporation. Hematoxylin-eosin and neutral resin were obtained from China Biyuntian Biotechnology Co., Ltd.

Instruments: Nikon optical microscope imaging system (Japan); embedding machine: HistoCore Arcadia (Leica); microtome: RM2235 (Leica); automatic staining machine: LEICA Autostainer ST5020; slide scanner: Hamamatsu NanoZoomer Digital Pathology (S210); analytical balance: METTLER Toledo ALT104; weighing scale: Changshu Shuangjie Testing Instrument Factory (T1000); surgical microscope: Luckbird XTS-4A; physiological recorder: RM6240E (Chengdu Instrument Factory)

Experimental Animals: SPF-grade male Sprague-Dawley (SD) rats were obtained from the Guangdong Provincial Medical Experimental Animal Center (License No.: SCXK (Yue) 20180002), with an average weight of 150±5 grams. The Guangdong Pharmaceutical University Experimental Animal Center holds the experimental unit license number SYXK (Yue) 2017-0125. The animal room is maintained at a temperature of 20-25° C. and a humidity level of 55%-65%, with a 12-hour light-dark cycle. The animals have unrestricted access to food and water.

### Experimental Methods

Experimental Groups: SD rats were randomly divided into five groups, with 15 rats in each group, as follows: Normal Control Group (Vehicle); PAH Model Group (MCT); PAH+Low-Dose Sildenafil Group (hereinafter referred to as the Low-Dose Original Drug Group); PAH+High-Dose Sildenafil Group (hereinafter referred to as the High-Dose Original Drug Group); and PAH+Tadalafil Gastro-Resistant Fast-Release New Formulation Solid Dispersion Tablets (hereinafter referred to as the Fast-Release Tablet Group).

### Animal Modeling:

After fully dissolving MCT in 1M dilute HCl, dilute the solution with double-distilled water. Then, adjust the pH to 7.2 using 3M NaOH to prepare a final concentration of 20 mg/ml of MCT solution for future use.

The normal control group received an intraperitoneal injection of saline at a dosage of 50 mg/kg, while the other groups were administered a single intraperitoneal injection of MCT at the same dosage of 50 mg/kg.

### Dosage and Administration:

Normal control group: no treatment administered.
PAH Model Group: No Treatment.

In the original low-dose drug group, Cialis was finely ground into a powder using a mortar and mixed with saline to prepare a freshly made solution of 0.4 mg/ml. This solution was administered by gavage at a dosage of 2 mg/kg/day.

In the original high-dose drug group, Cialis was finely ground into a powder using a mortar and mixed with saline to prepare a freshly made 1 mg/ml solution. This solution was administered by gavage at a dosage of 5 mg/kg/day.

The immediate-release tablet group consisted of tadalafil gastro-soluble immediate-release solid dispersion tablets, which were finely ground into a powder using a mortar. This powder was then mixed with saline to prepare a freshly made solution at a concentration of 0.4 mg/ml. The solution was administered by gavage at a dosage of 2 mg/kg/day.

The day of injection marks the beginning of the experiment. Treatment commenced 7 days after modeling, with administration occurring once daily for a total of 14 days.

### Experimental Indicators and Detection Methods

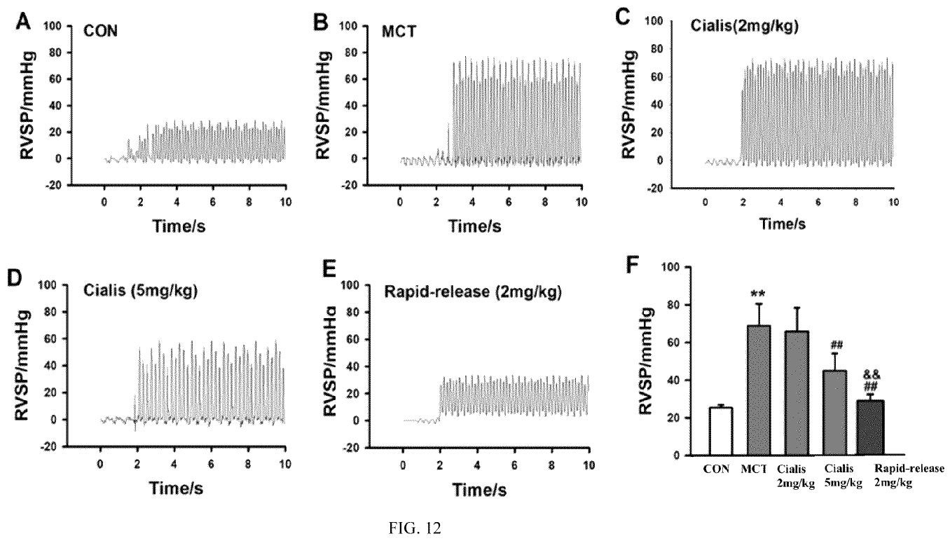

Right Ventricular Systolic Pressure (RVSP) in PAH Rats: Three weeks after the commencement of the experiment, RVSP data for each group of rats were collected using the RM-6240E multi-channel physiological recorder. The results are presented in Appendix FIG. 12.

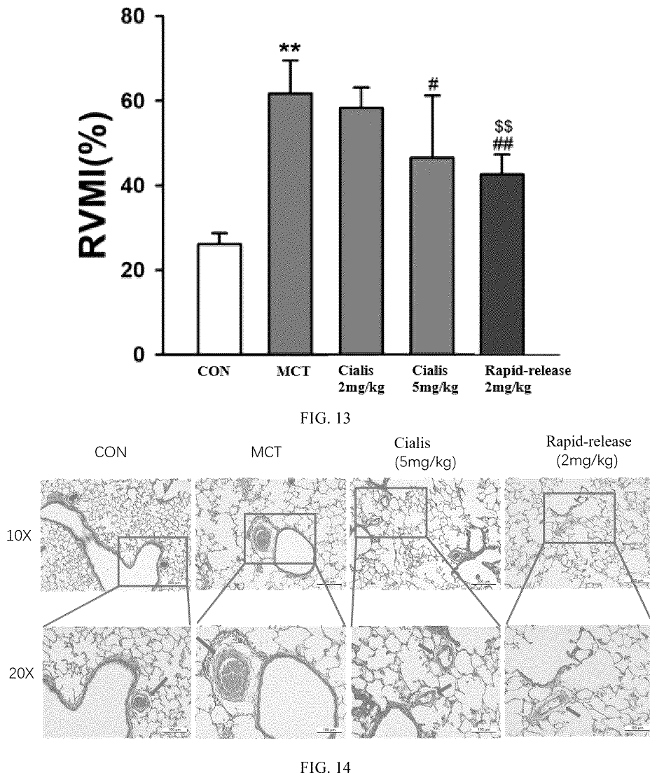

Right Ventricular Hypertrophy Index (RVMI) in PAH Rats: Three weeks after the initiation of the experiment, the animals were anesthetized for hemodynamic testing using the RM-6240E multi-channel physiological recorder. Following the pressure measurements, the animals were euthanized, and the heart and lung tissues were promptly dissected. Using ophthalmic scissors, the right and left atria, along with the major blood vessels, were excised at the atrioventricular junction. The right ventricle (RV) and the left ventricle plus septum (LV+S) were then separated. After blotting away excess moisture with filter paper, the masses were weighed individually, and the RV/(LV+S) ratio was calculated to determine the RVMI. The results are presented in Appendix FIG. 13.

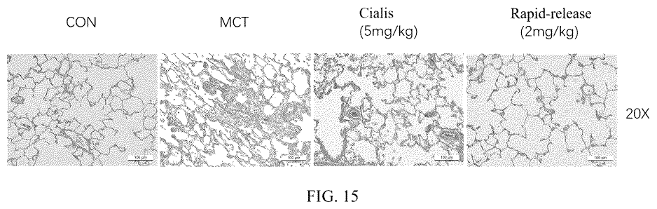

Pathological Observation of Pulmonary Small Arteries and Pulmonary Interstitial Fibrosis in PAH Rats: Three weeks after the initiation of the experiment, the rats were treated with heparin (50 IU/100 g, intraperitoneal injection) for 5 minutes, followed by the administration of a 20% uratan solution (0.5 ml/100 g) to anesthetize the animals. Blood pressure parameters were measured and recorded. The heart was dissected, the right ventricular wall was separated, and the left ventricle along with the interventricular septum was isolated. After blotting with filter paper, these tissues were weighed separately to calculate the right ventricular hypertrophy index. The lungs were dissected, the right lung was ligated, and the left lung lobe was perfused with fixative (4% paraformaldehyde) before being placed in fixative for further fixation. The samples underwent gradient dehydration with ethanol, clearing with xylene, embedding in paraffin, sectioning (4 μm), and staining with hematoxylin-eosin (HE). After mounting with neutral gum, the samples were observed under a microscope. Different sections were randomly examined in six fields of view to assess changes in the thickness of the media in small arteries, comparing them with the control group to determine histological changes in the lung tissue of each group of animals. An image acquisition and analysis system were utilized to observe changes in the media thickness and outer diameter of pulmonary small arteries (diameter 50-150 μm) in each group of rats, with results presented in FIGS. 14-15.

### Experimental Results

RVSP in PAH Rats: FIG. 12 illustrates the effects of various doses of Cialis and gastro-resistant fast-release solid dispersion tablets of tadalafil on the RVSP of rats with PAH after 14 days of treatment. The results indicate that, compared to the normal control group, the RVSP in the PAH model group was significantly elevated. In contrast to the model group, the RVSP in the low-dose Cialis group did not show a significant decrease, while the high-dose group demonstrated a significant reduction in RVSP. Additionally, the fast-release tablet group exhibited a notable decrease in RVSP compared to the PAH model group; furthermore, the reduction in RVSP in the fast-release tablet group was more pronounced than that observed in the high-dose Cialis group.

RVMI in PAH Rats: FIG. 13 illustrates the effects of various doses of Cialis and gastro-resistant fast-release solid dispersion tablets of tadalafil on the RVMI of rats with PAH after 14 days of treatment. The results indicate that, compared to the normal control group, the RVMI in the PAH model group was significantly elevated. In contrast to the model group, the RVMI in the low-dose Cialis group did not show a significant decrease, while the high-dose group exhibited a notable reduction and improvement in RVMI. Furthermore, the fast-release tablet group demonstrated a significant decrease in RVMI compared to the PAH model group; additionally, the reduction in RVMI in the fast-release tablet group was more pronounced than that observed in the high-dose Cialis group.

Pathological Observations of Pulmonary Small Arteries and Interstitial Fibrosis in Rats with PAH: The changes in media thickness and lumen area of the pulmonary small arteries were examined across different groups of rats. FIG. 14 presents standard images of H&E staining of pulmonary small arteries from various groups of rats treated with different doses of Cialis and tadalafil gastro-resistant fast-release solid dispersion tablets after 14 days, magnified at 100× and 200×. Compared to the normal control group, the media thickness of the pulmonary small arteries in the rats of the PAH model group was significantly increased, while the lumen area was reduced. In contrast, both the high-dose Cialis group and the fast-release tablet group demonstrated significant improvements in the media thickness and lumen area of the pulmonary small arteries when compared to the model group. The fast-release tablet group demonstrated a more pronounced effect in reducing intimal hyperplasia in the pulmonary small arteries compared to the high-dose Cialis group. FIG. 15 displays standard images of Masson's trichrome staining of lung tissue from different groups of rats treated with various doses of Cialis and tadalafil gastro-resistant fast-release solid dispersion tablets after 14 days, magnified at 200×. Compared to the normal control group, collagen deposition in the interstitial tissue of the lungs in the PAH model group rats was significantly increased. In contrast, both the high-dose Cialis group and the fast-release tablet group exhibited a significant reduction in collagen deposition in lung tissue compared to the model group. Notably, the fast-release tablet group demonstrated a more pronounced effect in improving collagen deposition than the high-dose Cialis group.

### 3. Pulmonary Fibrosis Rat Experiment

### Experimental Materials

Reagents: Paraquat was obtained from Sigma Chemical Company, USA. The tadalafil enteric-coated immediate-release solid dispersion 2 mg tablets were prepared as described in Example 5. Cialis was purchased from JD Pharmacy (Batch number: D448743). Isoflurane, pentobarbital sodium anesthetic, and formalin were acquired from China National Pharmaceutical Group Corporation.

Instruments include a Nikon optical microscope imaging system from Japan; a HistoCore Pearl tissue dehydrator from Leica; a HistoCore Arcadia embedding machine from Leica; an RM2235 microtome from Leica; a LEICA Autostainer ST5020 automatic staining machine; a Hamamatsu NanoZoomer Digital Pathology (S210) slide scanner; a METTLER Toledo ALT104 analytical balance; a Changshu Shuangjie Testing Instrument Factory T1000 weighing scale; a Jwilch electric heating blanket from China; and a Luckbird XTS-4A surgical microscope.

Experimental Animals: Male SD rats, SPF grade, weighing between 230 and 260 grams, were obtained from Guangdong Weitong Lihua Experimental Animal Technology Co., Ltd., under production license number [SCXK (Yue) 2022-0063]. The animals were housed at the Animal Experiment Center of South China University of Technology, with license number [SYXK (Yue) 2017-0178]. The research protocol received approval from the Animal Experiment Ethics Committee of South China University of Technology (2018042) and complied with international standards for temperature, humidity, and light control systems.

### Experimental Methods

A total of 40 male SD rats were randomly assigned to five groups based on body weight, with eight rats in each group: Normal group, Paraquat model group (PQ), Paraquat+low-dose Cialis group (hereinafter referred to as the low-dose original drug group), Paraquat+high-dose Cialis group (hereinafter referred to as the high-dose original drug group), and Paraquat+tadalafil gastro-resistant fast-release solid dispersion tablet (hereinafter referred to as the fast-release tablet group).

Animal Modeling: With the exception of the normal group, which received a single intraperitoneal injection of saline, the other four groups of rats were administered a single intraperitoneal injection of PQ (30 mg/kg) to establish a pulmonary fibrosis rat model. Two hours later, the Cialis treatment group and the fast-release tablet treatment group were gavaged with different doses of Cialis or fast-release tablets, respectively. Meanwhile, the normal group and the model group received daily gavage of an equal volume of saline, continuing the treatment for four weeks.

### Dosage and Administration Method

Administer physiological saline via gavage based on body weight, with a dosage of 1 mL per 100 g per day.

Original Research Drug Low Dose Group: Cialis is ground into a fine powder using a mortar and mixed with physiological saline to prepare a fresh solution of 0.2 mg/mL, which is administered by gavage at a dosage of 1 mg per kg per day.

Original Research Drug High Dose Group, Cialis is ground into a fine powder using a mortar and mixed with physiological saline to prepare a fresh solution of 1 mg/mL. This solution is administered by gavage at a dosage of 5 mg per kilogram per day.

Immediate Release Tablet Group: Tadalafil gastro-soluble immediate-release solid dispersion tablets are ground into a fine powder using a mortar and mixed with physiological saline to prepare a fresh solution at a concentration of 0.2 mg/mL. This solution is administered by gavage at a dosage of 1 mg per kg per day.

Each group of rats began oral administration on the day of modeling, receiving treatment once daily for a total of four weeks. On day 29, the rats were anesthetized and euthanized via intraperitoneal injection of 2.5% tribromoethanol (100 g/mL). The abdominal cavity was opened, the abdominal aorta was severed, and the heart was allowed to cease beating. The heart and lungs were then excised, and the surface moisture was gently absorbed with dry filter paper (three times) before weighing and photographing. Hemostatic clamps were employed to ligate the right pulmonary artery and vein, as well as the right main bronchus. The left lung was fixed by perfusion with 4% paraformaldehyde, and after ligation with cotton thread, it was immersed in 4% paraformaldehyde solution for fixation, to be used for subsequent paraffin sectioning.

### Experimental Indicators and Detection Methods

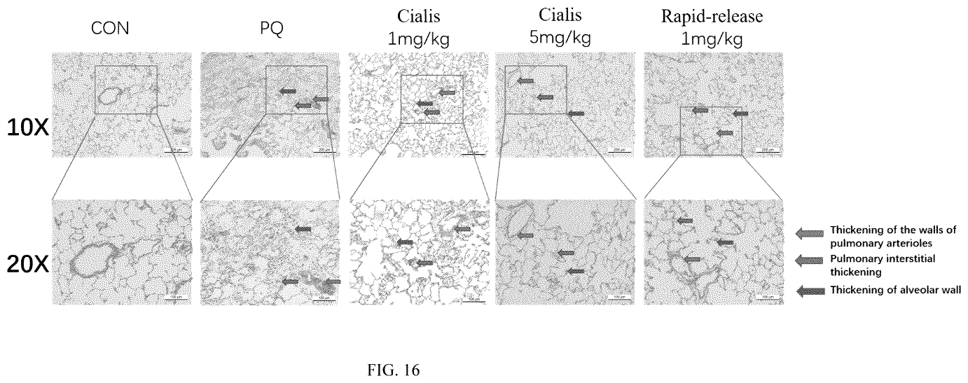

Lung Tissue Pathological Observation: Lung tissues from each group were fixed for over 24 hours, followed by dehydration using varying concentrations of ethanol, transparency treatment with xylene, embedding in paraffin, and sectioning into continuous slices (4 μm) of the largest longitudinal lung section. After baking the slides, they were deparaffinized with xylene and stained with HE. The samples were then dehydrated, cleared, and finally mounted with neutral resin. The pathological structure and morphology of the lung tissue were examined under an optical microscope, and representative fields were photographed, as illustrated in Appendix FIG. 16.

Lung Tissue Masson Trichrome Staining: The lung tissue slides from each group were baked and then deparaffinized using xylene, followed by dehydration with varying concentrations of ethanol. The samples were stained overnight with potassium dichromate, rinsed with running water, and then stained with iron hematoxylin. After differentiation, the samples were rinsed again with running water. Following the staining with acid fuchsin, the slides were continuously rinsed with running water. Afterward, the slides were directly stained with aniline blue solution without prior washing. Following differentiation, the samples were routinely dehydrated, cleared, and mounted. Finally, once the mounting medium had dried, the samples were examined under an optical microscope to assess the deposition of collagen fibers in the lung tissue, and representative fields were photographed, as shown in Appendix FIG. 17.

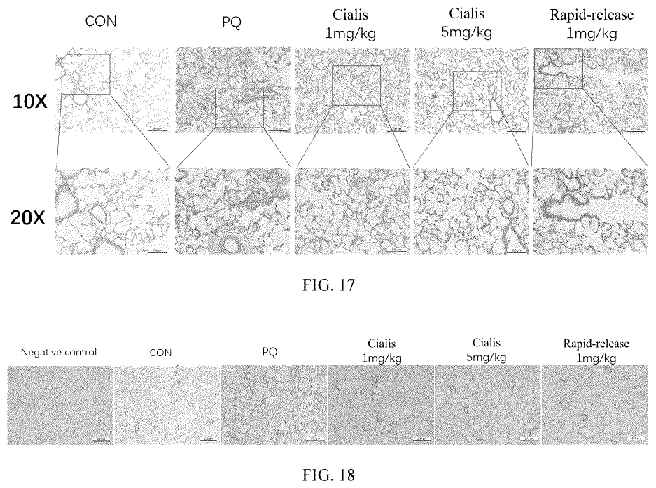

Immunohistochemical Staining of α-SMA in Lung Tissue: The tissue sections were routinely dewaxed in distilled water, washed three times with phosphate-buffered saline (PBS), and subjected to antigen retrieval. The SP-9001 reagent from Zhongshu Gold Bridge was applied to block endogenous peroxidase for 10 minutes at room temperature. Goat serum was used for blocking at room temperature for 1 hour. The primary antibody, α-SMA (1:100), was incubated overnight at 4° C. The following day, the sections were washed three times with PBS, and biotin-labeled goat anti-rabbit IgG polymer was added and incubated at room temperature for 15 minutes, followed by three washes with PBS. A working solution of horseradish peroxidase-labeled streptavidin was then added and incubated at room temperature for an additional 15 minutes, followed by three washes with PBS. The DAB chromogenic reaction was performed, followed by hematoxylin counterstaining for 3 minutes, differentiation, bluing, dehydration, clearing, and mounting. The results are presented in FIG. 18.

### Experimental Results

Lung Tissue Structural Damage in Rats with Pulmonary Fibrosis: FIG. 16 presents standard H&E staining images of lung tissue from various groups of rats treated with different doses of Cialis and tadalafil gastro-resistant fast-release solid dispersion tablets after 28 days, magnified at 100× and 200×. The results indicate that the normal group of rats displayed typical alveolar morphology, characterized by a regular arrangement, clear alveolar septa, and no significant infiltration of inflammatory cells or fibrotic proliferation. In contrast, the PQ model group exhibited severely damaged lung tissue structure under microscopic examination, with marked thickening of the alveolar walls, alveolar septa, and the walls of the pulmonary arterioles, accompanied by pronounced infiltration of inflammatory cells. However, after treatment with various doses of Cialis and gastro-resistant fast-release solid dispersion tablets containing tadalafil, the structural damage in the lung tissue of the rats showed significant improvement. Compared to the model group, the alveolar morphology appeared more intact, with notable improvements in the thickening of the alveolar walls, alveolar septa, and the walls of the pulmonary arterioles, accompanied by a marked reduction in inflammatory cell infiltration. The treatment effect of the low-dose fast-release tablets was comparable to that of the high-dose Cialis.

Collagen Deposition in the Lung Tissue Interstitium of Pulmonary Fibrosis Rats: FIG. 17 presents standard Masson trichrome staining images of lung tissue from various groups of rats treated with different doses of Cialis and tadalafil gastro-resistant fast-release solid dispersion tablets after 28 days of treatment for pulmonary fibrosis, magnified at 100× and 200×. The results indicate that the lung tissue structure in the normal group of rats is generally intact, exhibiting minimal blue collagen deposition, with only a small amount of collagen fibers present in the alveolar septa. The PQ model group demonstrates fibrotic damage in lung tissue, characterized by severe fibrosis that results in the loss of alveolar structure and a significant accumulation of striped blue collagen fibers within the thickened alveolar walls of the fibrotic regions. Following treatment with various doses of Cialis and gastro-resistant fast-release solid dispersion tablets of tadalafil, collagen deposition in the lung tissue interstitium was significantly reduced. Compared to the low-dose Cialis group, the high-dose Cialis exhibited a more pronounced inhibitory effect on collagen deposition, while the treatment effect of the low-dose fast-release tablets was comparable to that of the high-dose Cialis.

Expression and Distribution of the Epithelial-Mesenchymal Transition Marker α-SMA in Lung Tissue of Fibrotic Rats: FIG. 18 presents standard immunohistochemical images of α-SMA in the lung tissues of different groups of rats treated with various doses of Cialis and tadalafil gastro-resistant fast-release solid dispersion tablets after 28 days of treatment for pulmonary fibrosis, magnified at 100×. The results indicate that, compared to the normal group, the expression of the mesenchymal marker protein α-SMA significantly increased in the lung tissue of the PQ model group rats. After treatment with Cialis or fast-release tablets, the expression and distribution of the fibrotic marker molecule α-SMA significantly decreased. Compared to the low-dose Cialis group, the high-dose Cialis demonstrated a more pronounced inhibitory effect on α-SMA expression. In contrast, the inhibitory effect of the low-dose fast-release tablets was comparable to that of the high-dose Cialis.

### 4. Liver Fibrosis Mouse Experiment

### Experimental Materials

Reagents: Corn oil (C116025) was purchased from Shanghai Aladdin Company; CCl4 (XW0056232), tribromoethanol, and formaldehyde were obtained from China National Pharmaceutical Group Corporation. Tadalafil enteric-coated fast-release solid dispersion 2 mg tablets were prepared as described in Example 5. Cialis was purchased from JD Pharmacy (Batch number: D448743). Mouse anti-α-SMA (CST 19245) was acquired from American CST Company, while the hematoxylin and eosin staining kit (C0105) was sourced from Shanghai Biotime Biotechnology Co., Ltd. The Sirius Red staining solution (Solarbio, G1470) was purchased from Beijing Leigen, and the immunohistochemistry kit was obtained from Zhongshan Golden Bridge (SP9001).

Instruments include a Nikon optical microscope imaging system from Japan; a HistoCore Pearl tissue dehydrator from Leica; a HistoCore Arcadia embedding machine from Leica; an RM2235 microtome from Leica; a LEICA Autostainer ST5020 automatic stainer; a Hamamatsu NanoZoomer Digital Pathology (S210) slide scanner; a METTLER Toledo ALT104 analytical balance; and a Changshu Shuangjie Testing Instrument Factory T1000 weighing scale.

Experimental Animals: SPF-grade male C57BL/6 mice (20-22 grams) were purchased from Hunan Silek Jingda Laboratory Animal Co., Ltd. (Production License No.: SCXK (Xiang) 2016-0002). The animals were housed at the Animal Experiment Center of South China University of Technology (SYXK (Yue) 2017-0178), and the research protocol received approval from the Animal Experiment Ethics Committee of South China University of Technology.

### Experimental Methods

A total of 20 male C57BL/6 mice were randomly assigned to four groups based on body weight, with five mice in each group: the Normal group, the Carbon Tetrachloride Model group (CCl₄), the CCl₄+Original Drug Treatment group (hereinafter referred to as the Original Drug group), and the CCl₄+Tadalafil Gastro-resistant Fast-release New Formulation Solid Dispersion Tablet group (hereinafter referred to as the Fast-release Tablet group).

Animal Modeling: With the exception of the Normal group, the other three groups of mice were intraperitoneally injected with a 20% CCl₄ corn oil solution (5 ml/kg) twice a week for a duration of 8 weeks to establish a mouse model of liver fibrosis.

### Dosage and Administration Method

The model group received physiological saline administered via gavage, adjusted according to body weight.

The active ingredient of Cialis was finely ground into a powder using a mortar and mixed with physiological saline to prepare a freshly made 0.5 mg/ml solution. This solution was administered via gavage at a dosage of 2.5 mg/kg/day.

Immediate Release Tablet Group: Tadalafil gastro-soluble immediate-release solid dispersion tablets were ground into a powder using a mortar and mixed with physiological saline to prepare a 0.2 mg/ml solution (freshly prepared for use), which was administered via gavage at a dosage of 1 mg/kg/day.

After four weeks of modeling, the treatment groups of mice began receiving gavage of Cialis or immediate-release tadalafil tablets, while the normal control group and model group mice were gavaged daily with an equal volume of physiological saline, continuing for an additional four weeks. At the end of the modeling period, the mice were anesthetized and euthanized using 1.25% tribromoethanol (250 mg/kg). The abdominal cavity was opened, and the liver was exposed and photographed. The liver was then removed and fixed in a 4% paraformaldehyde solution for subsequent paraffin sectioning.

### Experimental Indicators and Detection Methods

Liver Tissue: Liver tissues from each group were fixed for over 24 hours, followed by dehydration through a series of ethanol concentrations, clearing with xylene, infiltration with paraffin, and embedding in paraffin blocks. Continuous sections (4 μm) were cut from the largest surface, baked, and then deparaffinized with xylene. HE staining was performed, followed by dehydration and clearing. Finally, the slides were mounted with neutral resin. The pathological structure and morphology of the liver tissue were observed under an optical microscope, and images of the fields of view were captured.

Liver Tissue Masson Trichrome Staining: Liver tissue slides from each group were deparaffinized using xylene, dehydrated with varying concentrations of ethanol, and stained overnight with potassium dichromate. After rinsing with running water, the slides were stained with hematoxylin, differentiated, and rinsed again with running water. Following the acid fuchsin staining, the slides were continuously rinsed with running water. After staining with phosphomolybdic acid, the slides were directly placed in an aniline blue staining solution without prior washing. Following differentiation, the slides were routinely dehydrated, cleared, and mounted. Finally, once the mounting medium had dried, the liver tissue was observed and evaluated under an optical microscope for collagen fiber deposition, and images of the selected fields were captured.

Sirius Red Staining of Liver Tissue: After deparaffinization of liver tissue slides from each group, the slides were stained with Sirius Red solution for 5 minutes. Subsequently, the slides were immersed in anhydrous ethanol for dehydration, followed by xylene for transparency. Finally, the slides were mounted with neutral gum, and once the mounting medium dried, the liver tissue was examined and evaluated under an optical microscope for collagen fiber deposition, with selected fields photographed.

Liver Tissue α-SMA Immunohistochemical Staining: The tissue sections were routinely dewaxed in distilled water, washed three times with PBS, and subjected to antigen retrieval. Subsequently, SP-9001 reagent was applied to block endogenous peroxidase activity, and the samples were incubated at room temperature for 10 minutes. Goat serum was utilized for blocking at room temperature for 1 hour. The primary antibody, α-SMA (1:100), was incubated overnight at 4° C. The following day, after washing the sections three times with PBS, biotin-labeled goat anti-rabbit IgG polymer was added and incubated at room temperature for 15 minutes, followed by three additional washes with PBS. A horseradish peroxidase-labeled streptavidin working solution was then added and incubated at room temperature for 15 minutes, followed by three washes with PBS. A DAB chromogenic reaction was performed, followed by hematoxylin counterstaining for 3 minutes, differentiation, bluing, dehydration, clearing, and mounting.

### Experimental Results

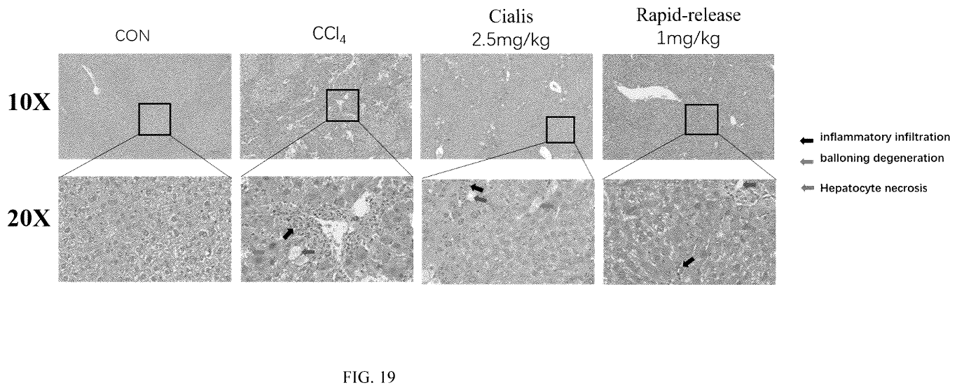

Liver Tissue Structural Damage in Mice with Liver Fibrosis: FIG. 19 presents standard H&E staining images of liver tissue from various groups of mice treated with a new formulation of solid dispersion tablets containing Sildenafil and Tadalafil after four weeks, magnified at 100× and 200×. The results indicate that in the normal control group, the liver cell morphology appears normal, with cells arranged in a regular pattern and no infiltration of inflammatory cells; the liver lobule structure remains largely intact. In contrast, the CCl₄ model group exhibits severe liver damage, characterized by necrosis of liver cells, significant infiltration of inflammatory cells, and destruction of the liver lobule structure. In contrast, the liver tissue damage in the PDE5 inhibitor treatment group shows significant improvement. Compared to the Sildenafil group, the fast-release tablet group exhibits a more pronounced therapeutic effect, evidenced by a noticeable reduction in the number of necrotic liver cells, a generally intact liver lobule structure, and a decrease in the degree of inflammatory cell infiltration.

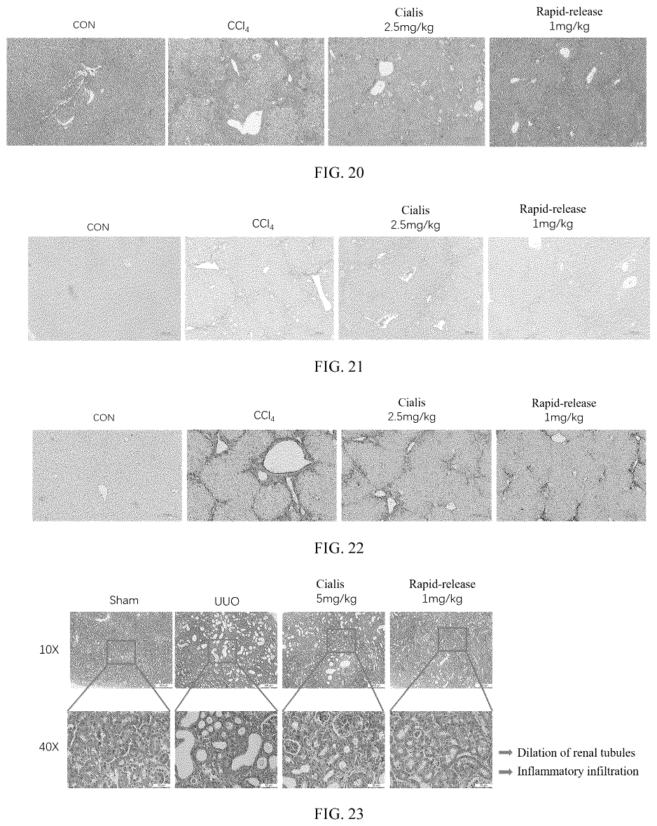

Collagen Deposition in Liver Tissue of Mice with Liver Fibrosis: FIGS. 20 and 21 present standard images of Masson's trichrome staining and Sirius Red staining of liver tissues from various groups of mice treated with a novel formulation of solid dispersions of sildenafil and tadalafil for four weeks, magnified at 200×. The results indicate that the liver tissue structure in the normal control group remains intact, exhibiting minimal blue or red positive staining. Additionally, the areas of collagen positive staining are primarily located around the portal regions. In the CCl₄ model group, there is a noticeable deposition of striped blue collagen fibers and Sirius Red-positive areas in the portal regions, indicating a significant amount of collagen accumulation and the presence of portal-portal bridging. In the PDE5 inhibitor treatment group, collagen deposition in the portal areas is significantly reduced, with no portal-portal bridging observed. Furthermore, the treatment effect of the immediate-release tablets is more pronounced compared to the sildenafil group.

Expression and Distribution of the Epithelial-Mesenchymal Transition Marker α-SMA in the Liver Tissue of Fibrotic Mice: FIG. 22 presents standard immunohistochemical images of α-SMA in the liver tissues of various groups of mice treated with a novel solid dispersion tablet formulation of Sildenafil and Tadalafil for four weeks, magnified 100 times. The results indicate that, compared to the normal control group, the expression and distribution of α-SMA in the liver tissue of the CCl₄ model group mice were significantly upregulated. In contrast, treatment with PDE5 inhibitors markedly reduced the levels of α-SMA expression and distribution, with the immediate-release tablets demonstrating a more pronounced inhibitory effect on α-SMA expression and distribution.

### 5. Kidney Fibrosis Mouse Experiment

### Experimental Materials

Reagents: The tadalafil gastro-resistant fast-release solid dispersion 2 mg tablets were prepared as described in Example 5. Cialis was purchased from JD Pharmacy (Batch No.: D448743). Penicillin, xylene, and anhydrous ethanol were obtained from Sigma in the USA. Mouse anti-α-SMA (CST 19245) was sourced from CST in the USA, while fibronectin (FN) was purchased from Abcam. The hematoxylin and eosin staining kit (C0105) was acquired from Shanghai Biyuntian Biotechnology Co., Ltd., and the immunohistochemistry kit was obtained from Zhongshan Golden Bridge (SP9001).

Instruments include a Nikon optical microscope imaging system from Japan; a HistoCore Pearl tissue dehydrator from Leica; a HistoCore Arcadia embedding machine from Leica; an RM2235 microtome from Leica; a LEICA Autostainer ST5020 automatic stainer; a Hamamatsu NanoZoomer Digital Pathology (S210) slide scanner; a METTLER Toledo ALT104 analytical balance; and a Changshu Shuangjie Testing Instrument Factory T1000 weighing scale.

Experimental Animals: SPF-grade male C57BL/6 mice (22-23 grams) were purchased from Hunan Sileke Jingda Experimental Animal Co., Ltd. (License No. 43072711100365523) and housed at the Experimental Animal Center of Guangdong Pharmaceutical University (License No. SYXK (Yue) 2017-0125). This experimental protocol strictly adheres to the animal care guidelines established by the Animal Ethics Committee of Guangdong Pharmaceutical University (Approval No. gdpulacspf2017027).

### Experimental Methods

A total of 20 male C57BL/6 mice were randomly assigned to four groups based on their body weight, with five mice in each group: the sham surgery group (Sham group), the unilateral ureteral obstruction model group (UUO), the unilateral ureteral obstruction plus sildenafil treatment group (hereinafter referred to as the original drug group), and the unilateral ureteral obstruction plus tadalafil gastro-resistant fast-release solid dispersion tablet group (hereinafter referred to as the fast-release tablet group).

Animal Modeling: Prior to the surgery, the mice were weighed, and 1.25% tribromoethanol was administered intraperitoneally to induce anesthesia. The fur on the left side of the abdomen was shaved, and after routine disinfection with 75% alcohol, the mice were positioned laterally on their right side and secured to the surgical table. A 0.5 cm diagonal incision was made on the left side of the abdomen, and the abdominal wall was opened layer by layer. Using curved forceps, the surrounding fat was gently displaced to expose and free the left kidney and ureter. The ureter was ligated at two points using 5-0 sutures and then transected in the middle. Following successful ligation, the abdominal cavity was irrigated with antibiotics, the layers were sutured back together, iodine was applied, and the mice were placed on a heating pad to facilitate recovery. The sham surgery group underwent the same procedure, but the ureter was only freed without ligation.

### Dosage and Administration Method

Administer physiological saline via gavage according to body weight.

The original drug group, Cialis, is ground into a fine powder using a mortar and mixed with physiological saline to prepare a fresh 1 mg/ml solution. This solution is administered via gavage at a dosage of 5 mg/kg/day.

Immediate Release Tablet Group: Tadalafil gastro-soluble immediate-release solid dispersion tablets are ground into a fine powder using a mortar and mixed with physiological saline to prepare a 0.2 mg/mL solution (prepared fresh for use). This solution is administered via gavage at a dosage of 1 mg/kg/day.

Two hours after modeling, the treatment group mice were gavaged with either Cialis or immediate-release tadalafil tablets, while the sham surgery group and model group mice received daily gavage of an equal volume of physiological saline for a duration of 7 days. Following the modeling period, the mice were anesthetized and euthanized using 1.25% tribromoethanol (250 mg/kg). The affected kidneys were then removed and quickly rinsed in ice-cold PBS to eliminate blood stains. The extracted kidneys were sectioned along the coronal plane; one portion was fixed in 4% paraformaldehyde, while another portion was frozen in liquid nitrogen for subsequent Western blot analysis.

### Experimental Indicators and Detection Methods

Renal Tissue Pathology Observation: Kidney tissues from each group were fixed for over 24 hours, followed by dehydration through varying concentrations of ethanol, clearing with xylene, infiltration with paraffin, and embedding in paraffin blocks. Continuous sections (4 μm) were cut from the largest surface, baked, and then deparaffinized with xylene. HE staining was performed, followed by dehydration and clearing, and the sections were finally mounted with neutral resin. The pathological structure and morphology of the renal tissue were observed under an optical microscope, and images of the fields of view were captured.

Kidney Tissue Masson Trichrome Staining: Kidney tissue slides from each group were dewaxed using xylene, dehydrated with varying concentrations of ethanol, and stained overnight with potassium dichromate. After rinsing with running water, the slides were stained with hematoxylin, differentiated, and rinsed again with running water. Subsequently, they were stained with acid fuchsin and continuously rinsed with running water. Following the staining with phosphomolybdic acid, the slides were directly placed in an aniline blue staining solution without prior washing. After differentiation, the slides were routinely dehydrated, cleared, and mounted. Finally, once the mounting medium had dried, the kidney tissue was examined and evaluated under an optical microscope for collagen fiber deposition, and images of the selected fields were captured.

Immunohistochemical Staining of α-SMA in Kidney Tissue: The tissue sections were routinely dewaxed in distilled water, washed three times with PBS, and subjected to antigen retrieval. The SP-9001 reagent was applied to block endogenous peroxidase activity, and the samples were incubated at room temperature for 10 minutes. Goat serum was utilized for blocking at room temperature for 1 hour. The primary antibody, α-SMA (1:100), was incubated overnight at 4° C. The following day, the samples were washed three times with PBS, and biotin-labeled goat anti-rabbit IgG polymer was added, followed by incubation at room temperature for 15 minutes, after which the samples were washed three times with PBS. A working solution of horseradish peroxidase-labeled streptavidin was added, and the samples were incubated at room temperature for 15 minutes. This was followed by three washes with PBS. DAB was utilized for color development, while hematoxylin was employed for counterstaining for 3 minutes. Subsequent steps included differentiation, bluing, dehydration, clearing, and mounting.

Western Blot Detection of Kidney Tissue: Begin by taking an appropriate amount of kidney tissue and adding it to RIPA lysis buffer that contains phosphatase and protease inhibitors. Grind the tissue thoroughly, then centrifuge at 4° C. at 1200×g for 20 minutes. Collect the supernatant to obtain the total protein from the tissue, and determine the protein concentration using the BCA method. After denaturing the protein, store it at −80° C. Prepare an SDS-PAGE gel and load 30-40 μg of total protein per well. Perform electrophoresis and transfer the proteins to a membrane. Block the membrane with 5% milk at room temperature for 2 hours, then incubate it with primary antibodies overnight at 4° C.: α-SMA (1:1000), FN (1:1000), and GAPDH (1:5000). Following this, incubate with a secondary antibody (1:5000) at room temperature for 1 hour. Finally, expose and develop the membrane using chemiluminescent reagents, and conduct semi-quantitative analysis using Image Lab software.

### Experimental Results

Kidney Tissue Structural Damage in Mice with Renal Fibrosis: FIG. 23 presents standard H&E staining images of kidney tissues from various groups of mice treated with a new formulation of solid dispersion tablets containing Cialis and tadalafil after 7 days, magnified at 100× and 400×. The results indicate that the sham-operated group (Sham) exhibited kidney tissue structures that were essentially normal, with no lesions, and the renal tubules were well-filled. In the UUO model group, the kidney tissue displayed dilated renal tubules, characterized by loose, swollen, and vacuolated epithelial cells, thickening of the glomerular basement membrane, significant infiltration of inflammatory cells in the renal interstitium, increased interstitial width, and pronounced fibrosis. Compared to the UUO model group, the PDE5 inhibitor treatment group exhibited reduced vacuolar degeneration in the renal tubules and a significant decrease in the severity of lesions. Furthermore, the therapeutic effect of the low-dose fast-release tablets was comparable to that of the high-dose Cialis.

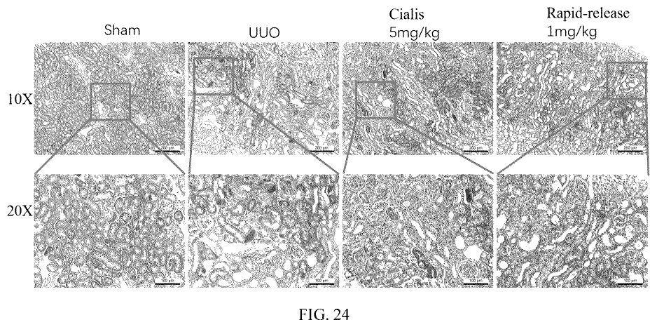

Collagen Deposition in Renal Tissue of Mice with Renal Fibrosis: FIG. 24 presents standard images of Masson's trichrome staining of kidney tissues from various groups of mice treated with a new formulation of solid dispersion tablets containing Cialis and tadalafil for 7 days, magnified at 100× and 200×. The results indicate that in the Sham group, the kidney tissue structure remains intact, exhibiting minimal red positive staining. The areas of collagen-positive staining are primarily located in the mesangial region and surrounding the capillaries of the renal tubular interstitium. In the UUO model group, there are significant Sirius Red-positive areas in the renal interstitium and tubules, indicating substantial collagen deposition. In comparison to the model group, the PDE5 inhibitor treatment group exhibited a marked reduction in collagen deposition within the tubular interstitium. Notably, the inhibitory effect of the low-dose immediate-release tablets on collagen deposition was comparable to that of the high-dose Cialis.

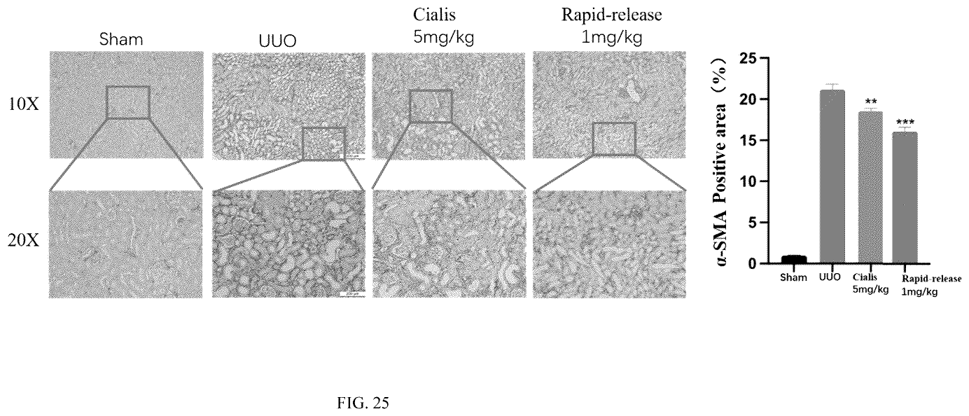

Expression and Distribution of Epithelial-Mesenchymal Transition Marker α-SMA in Renal Tissue of Fibrotic Mice: FIG. 25 presents standard immunohistochemical images of α-SMA in the renal tissues of various groups of mice treated with a novel formulation of solid dispersion tablets containing Cialis and Tadalafil for 7 days, magnified at 100× and 200×. The results indicate that, in comparison to the Sham group, the UUO model group mice demonstrated a significant upregulation in the expression and distribution of α-SMA in the affected renal tissue. In contrast, treatment with PDE5 inhibitors significantly reduced the levels of α-SMA expression and its distribution. Furthermore, the low-dose immediate-release tablets exhibited a more pronounced inhibitory effect on both the expression and distribution of α-SMA compared to the high-dose Cialis.

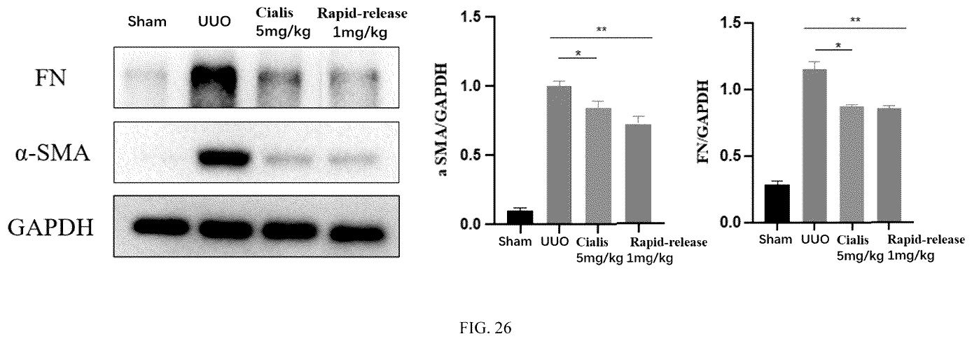

Expression of Fibrosis Marker Proteins in Renal Tissue of Mice with Renal Fibrosis: FIG. 26 illustrates the expression of fibrosis marker proteins α-SMA and FN in the renal tissue of mice with renal fibrosis following 7 days of treatment with a novel formulation of solid dispersion tablets containing Sildenafil and Tadalafil. The results demonstrate that, compared to the Sham group, the UUO model group mice exhibited a significant increase in the expression of α-SMA and FN in the affected renal tissue. However, treatment with both Sildenafil and the immediate-release tablets significantly reduced the expression levels of α-SMA and FN.

The experimental results presented above demonstrate that this application offers a novel dosage form of tadalafil as a solid dispersion tablet, characterized by gastric solubility and rapid release. This new formulation boasts advantages such as enhanced water solubility and elevated blood drug concentration. Furthermore, this application confirms that the gastric-soluble, rapid-release solid dispersion tablet of tadalafil can be utilized to treat pulmonary arterial hypertension, pulmonary fibrosis, liver fibrosis, and kidney fibrosis. It effectively reduces RVSP, alleviates right ventricular hypertrophy, improves pulmonary arterial proliferation, and diminishes the degree of fibrosis in pulmonary fibrosis lesions. It helps alleviate damage to alveolar structures and promotes the proliferation of small bronchi and pulmonary arterioles, significantly improving the pathological damage to vital organ tissue structures caused by drug or surgical interventions, while also delaying the progression of fibrosis. The gastro-soluble, fast-release solid dispersion tablet formulation of tadalafil presented in this application can be utilized to treat erectile dysfunction and myocardial hypertrophy. Furthermore, the new water-soluble formulation of tadalafil significantly enhances its solubility and blood concentration, which may reduce the required dosage for treatment, alleviate stress on the liver and kidneys, and lower the economic costs associated with disease management.

Clearly, the examples provided above are intended solely for illustrative purposes and do not restrict the methods of implementation. For those skilled in the relevant field, numerous other forms of alterations or modifications can be made based on the description provided. It is neither necessary nor feasible to provide an exhaustive list of all possible implementation methods. Obvious variations or modifications derived from this still fall within the protective scope of the invention.

1. A tadalafil gastric-soluble rapid-release solid dispersion, wherein the tadalafil has a structure depicted in the following formula (I), 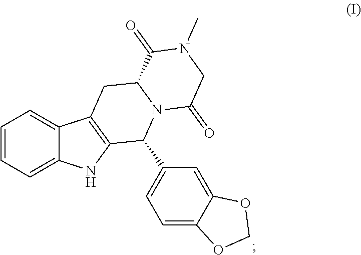 [SMILES: CN1CC(=O)N2[C@H](c3ccc4c(c3)OCO4)c3[nH]c4ccccc4c3C[C@@H]2C1=O]
an X-ray powder diffraction (XRPD) pattern of the solid dispersion is presented in FIG. 5, and there are no characteristic diffraction peaks at the following 2θ angles (±0.2°): 7.3°, 10.6°, 12.6°, 14.6°, 18.5°, 21.8°, and 24.3°, and the tadalafil exists in an amorphous form within the solid dispersion.

2. The tadalafil gastric-soluble rapid-release solid dispersion according to claim 1, wherein a TG-DSC spectrum of the tadalafil gastric-soluble rapid-release solid dispersion is presented in FIG. 6, and its differential scanning calorimetry (DSC) spectrum does not exhibit a characteristic melting absorption peak, and the tadalafil exists in an amorphous form within the solid dispersion.

3. The tadalafil gastric-soluble rapid-release solid dispersion according to claim 1, wherein the X-ray powder diffraction (XRPD) pattern of the tadalafil is illustrated in FIG. 3, and there are characteristic diffraction peaks at the following 2θ angles (±0.2°): 7.3°, 10.6°, 12.6°, 14.6°, 18.5°, 21.8°, and 24.3°.

4. The tadalafil gastric-soluble rapid-release solid dispersion according to claim 3, wherein a TG-DSC spectrum of the tadalafil is shown in FIG. 4, and the DSC spectrum displays a distinct endothermic peak at 305.4° C.

5. The tadalafil gastric-soluble rapid-release solid dispersion according to claim 1, the tadalafil gastric-soluble rapid-release solid dispersion comprises tadalafil, a polymer, and a surfactant;
preferably, a mass ratio of the tadalafil, polymer, and surfactant is 1:(4-6):(1-2).

6. The tadalafil gastric-soluble rapid-release solid dispersion according to claim 5, wherein the polymer comprises a mixture of one or more of the following: VA64, Soluplus, or HPMCP HP55.

7. The tadalafil gastric-soluble rapid-release solid dispersion according to claim 5, wherein the surfactant comprises a mixture of one or more of sodium dodecyl sulfate (SDS) or tocopheryl polyethylene glycol succinate (TPGS).

8. The tadalafil gastric-soluble rapid-release solid dispersion according to claim 6, wherein the tadalafil gastric-soluble rapid-release solid dispersion comprises the following components:
tadalafil, VA64, and TPGS are combined in a mass ratio of 1:4:1; or
tadalafil, HPMC, VA64, and SDS are combined in a mass ratio of 1:4:2:1; or
tadalafil, HPMC, and SDS are combined in a mass ratio of 1:6:1; or
tadalafil, HPMC, SDS, and TPGS are combined in a mass ratio of 1:6:1:1.

9. A method for preparing the tadalafil gastric-soluble rapid-release solid dispersion according to claim 1, wherein the method comprises the following steps:
mixing the specified amount of tadalafil, as indicated in formula (I), with a solvent to create an initial suspension;
adding the specified amounts of polymer and surfactant to the initial suspension to create a second suspension; and
spray-drying of the second suspension.

10. The method according to claim 9, wherein the solvent comprises a mixture of one or more of the following: acetonitrile, acetone, tetrahydrofuran, dichloromethane, or ethanol.

11. The method according to claim 9, wherein the method further comprises a step involving ultrasound-assisted dissolution or heat-assisted dissolution of the second suspension.

12. A tadalafil gastric-soluble rapid-release tablet, wherein the tadalafil gastric-soluble rapid-release tablet comprises the tadalafil gastric-soluble rapid-release solid dispersion according to claim 1.

13. The tadalafil gastric-soluble rapid-release tablet according to claim 12, wherein a mass content of tadalafil gastric-soluble rapid-release solid dispersion in the tablet ranging from 13.3% to 16.7% by weight.

14. A method for treating pulmonary arterial hypertension, myocardial hypertrophy, erectile dysfunction, or anti-fibrosis, comprising administering the tadalafil gastric-soluble rapid-release solid dispersion according to claim 1 to a subject in need.

15. The method according to claim 14, wherein the anti-fibrosis comprises at least one of the following: pulmonary fibrosis, liver fibrosis, or renal fibrosis.
